(19) United States
(12) Patent Application Publication
(10) Pub. No.: US 2022/0218883 A1
(43) Pub. Date: Jul. 14, 2022

(54) Implantable Device Coated by a Self-Assembled Monolayer and Therapeutic Agent

(71) Applicants: University of Pittsburgh - Of the Commonwealth System of Higher Education, Pittsburgh, PA (US); Duquesne University of the Holy Spirit, Pittsburgh, PA (US)

(72) Inventors: John J. Pacella, Bethel Park, PA (US); Ellen S. Gawalt, Pittsburgh, PA (US); Jared Romeo, Pittsburgh, PA (US)

(21) Appl. No.: 17/605,846

(22) PCT Filed: Apr. 24, 2020
(86) PCT No.: PCT/US2020/029832

§ 371 Date: Oct. 22, 2021

Related U.S. Application Data
(60) Provisional application No. 62/838,548, filed on Apr. 25, 2019.

(51) Int. Cl. A61L 31/16; A61L 31/08; A61K 31/7076

(52) U.S. Cl. CPC A61L 31/16; A61L 31/08; A61K 31/7076; A61L 2300/232; A61L 2300/416; A61L 2420/02; A61L 2300/80; A61L 2300/42

(57) ABSTRACT

After the claims, please insert a page containing the Abstract of the Disclosure which is attached hereto as a separately typed page.

### CROSS REFERENCE TO RELATED APPLICATION

This application claims priority to U.S. Provisional Patent Application No. 62/838,548 filed Apr. 25, 2019, entitled “Implantable Device Coated by a Self-Assembled Monolayer and Therapeutic Agent,” the disclosure of which is hereby incorporated by reference in its entirety.

### BACKGROUND OF THE INVENTION

### Field of the Invention

The present disclosure is generally directed to an implantable device and, more particularly, to an implantable device at least partially coated by a self-assembled monolayer and therapeutic agent covalently bonded to portions of the self-assembled monolayer.

### Description of Related Art

Implantable devices, such as endovascular devices and/or blood-contacting devices, are used for a variety of therapies, as are known in the art. For example, a common method for treating stenosed or aneurysmal vessels or other blocked passageways is to utilize an implantable expandable prosthesis or stent device. The prosthesis or stent device is an expandable structure configured to be deployed in a vessel or passageway in an expanded state to maintain patency or continuity of the vessel or passageway. Conventional stents are often formed from a framework of interconnecting members or struts, which can be arranged to form closed or open cells. Many stent designs are known and can include combinations of different types of framing structures, such as helical coils, meshes, lattices, or interconnected rings. Such framing structures can be made from, for example, stainless steel and/or cobalt chromium. Conventional stents can be covered or uncovered. The cover can be constructed from a biocompatible material, such as expanded polytetrafluoroethylene (ePFTE). In one common design, a stent can include a series of cylindrical rings aligned in a series along a central longitudinal axis. The rings can be fixedly secured to one another by a plurality of interconnecting members, such as longitudinally extending struts.

Placement of endovascular devices, such as stents, in the vascular system of a patient is known to cause a physiological response, such as thrombosis and local inflammation. For example, the blood-contacting surface of a metal stent can increase platelet aggregation and blood clot formation compared to a native, non-instrumented vessel wall (e.g., endothelium). Additionally, local trauma and vascular inflammation caused by stent implantation can result in vascular cell proliferation (also referred to as neointimal hyperplasia) into a lumen of the stent, resulting in restenosis and reduced blood flow. Various therapeutic agents can be provided to patients to reduce the immune response and/or to inhibit platelet aggregation and blood clot formation. For example, antiplatelet agents, such as ticagrelor, can be prescribed as antiplatelet therapies. Ticagrelor is taken orally and, therefore, must be taken in sufficient concentrations systemically to effect platelet inhibition at local stent site(s). This results in high systemic levels of ticagrelor and an associated bleeding risk.

Drug eluting stents formed from degradable biomaterials can be used for preventing neointimal hyperplasia. Current drug eluting stent systems can comprise a slowly degrading polymer, which allows a drug to seep into underlying tissues over a predetermined time period, such as a number of hours or days. Some drug eluting stents may also comprise a micro-textured surface that promotes direct adsorption of the drugs.

### SUMMARY OF THE INVENTION

Current methods for providing therapies for implantable devices, such as antiplatelet therapies, suffer from a number of deficiencies, as described herein. For example, providing medication orally or by injection does not target specific tissues or regions of the body, such as a wound site caused by deployment of an implantable device. Instead, in order to ensure that target tissues are exposed to a therapeutically effective concentration of a drug, the drug must be provided in sufficient amounts to provide system-wide efficacy. Accordingly, the patient may be exposed to systemic effects of the drug even when only targeted treatment is needed.

Drug eluting stents also may not provide targeted therapy when the implantable device is provided in proximity to dynamic tissues, such as flowing blood. In particular, the flowing blood carries away the drug eluted from the implantable device, meaning that the eluted drug does not collect in sufficiently-high concentrations in proximity to the implanted device. In order to account for drug carried away by blood flow, the implanted device may be configured to elute a sufficient amount of drug for system-wide efficacy.

The implantable medical devices and coatings disclosed herein are configured to address these issues by immobilizing therapeutic agents to surfaces of implantable devices. The immobilized therapeutic agents are configured to provide targeted therapies, such as antiplatelet therapies, directly to the tissues surrounding the implantable device for extended periods, such as for an entire useful life of the implantable device.

Beneficially, by immobilizing the therapeutic agent(s) on the implanted device, a level of therapeutic efficacy for tissues surrounding the implantable device can be achieved without needing to provide a sufficient amount of the drug for achieving system-wide efficacy to the patient. Accordingly, in one example, the medical devices and coatings disclosed herein avoid the need for antiplatelet therapy in order to prevent vascular interventions. Instead, the therapeutic agent(s) bonded and immobilized on the implantable medical device provide antiplatelet effects for tissues surrounding the implanted device, thereby avoiding the need to provide antiplatelet therapy with system-wide efficacy.

According to an aspect of the disclosure, an implantable device comprises a body configured to be implanted within a body of a patient and a self-assembled monolayer. The self-assembled monolayer comprises molecules comprising a first portion (moiety) bonded to a surface of the body, a second portion (moiety) opposite the first portion, and a linkage portion (moiety) extending between the first portion and the second portion. The implantable device further comprises a therapeutic agent comprising at least one therapeutic molecule covalently bonded to the second portion of the molecules of the self-assembled monolayer.

According to another aspect of the disclosure, a method of deploying an implantable device comprises advancing an implantable device through a vascular system of the patient to a preselected deployment site through a delivery catheter; and extending the implantable device from a distal end of the delivery catheter, thereby causing the implantable device to expand from a contracted state to a deployed state. The implanted device comprises a body configured to be implanted within a body of a patient and a self-assembled monolayer. The self-assembled monolayer comprises molecules comprising a first portion (moiety) bonded to a surface of the body, a second portion (moiety) opposite the first portion, and a linkage portion (moiety) extending between the first portion and the second portion. The implanted device further comprises a therapeutic agent comprising at least one therapeutic molecule covalently bonded to the second portion of the molecules of the self-assembled monolayer.

According to another aspect of the disclosure, a method of preparing an implantable device coated by a therapeutic agent comprises: preparing a body portion of an implantable device, which is configured to be blood contacting when implanted; exposing surfaces of the body of the implantable device to a solution containing molecules configured to form a self-assembled monolayer on the surfaces of the implantable device; and immersing the coated device comprising the self-assembled monolayer in a solution containing a therapeutic agent comprising at least one site configured to covalently bond to the at least one site of the self-assembled monolayer layer.

According to another aspect of the disclosure a method of deploying an implantable device comprises advancing an implantable device formed according to the previously described method through a vascular system of the patient to a preselected deployment site through a delivery catheter; and extending the implantable device from a distal end of the delivery catheter, thereby causing the implantable device to expand from a contracted state to a deployed state.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

FIG. 1A is a perspective view of an implantable device, according to an aspect of the present disclosure;

FIG. 1B is a cross sectional view of a portion of an elongated tine of the implantable device of FIG. 1A;

FIG. 1C is a perspective view of another embodiment of an implantable device in a deployed position, according to an aspect of the present disclosure;

FIG. 2A is a schematic drawing showing a coating comprising a self-assembled monolayer on a surface of an implantable device, according to an aspect of the disclosure;

FIG. 2B is a schematic drawing showing a coating comprising a self-assembled monolayer on a surface of an implantable device comprising two therapeutic agents, according to an aspect of the disclosure;

FIGS. 2C and 2D are schematic drawings showing coatings comprising mixed self-assembled monolayers on a surface of an implantable device, according to an aspect of the disclosure;

FIG. 3 is a flow chart of steps for forming a coating comprising a self-assembled monolayer and/or mixed self-assembled monolayer on a surface of an implantable device, according to an aspect of the disclosure;

FIG. 4 is a reaction scheme for immobilization of ticagrelor on a monolayer of 16-carboxylhexadecylphosphonic acid, according to an aspect of the disclosure;

FIG. 5 shows a comparison of a chemical structure of 2-phenoxyethanol and ticagrelor;

FIG. 6A is a reaction scheme for a Mitsunobu reaction for immobilization of 2-phenoxyethanol, according to an aspect of the disclosure;

FIG. 6B is a reaction scheme for a Mitsunobu reaction for immobilization of ticagrelor, according to an aspect of the disclosure;

FIG. 7 is a spectral graph obtained by DRIFT spectroscopy for a mixed monolayer comprising 16-carboxyhexadecylphosphonic acid and tetradecylphosphonic acid;

FIG. 8 is a spectral graph obtained by DRIFT spectroscopy for a self-assembled 12-aminododecylphosphonic acid monolayer after immobilization of 2-phenoxyethanol to the monolayer;

FIG. 9 is a spectral graph obtained by DRIFT spectroscopy for a self-assembled 12-aminododecylphosphonic acid monolayer after immobilization of ticagrelor to the monolayer;

FIGS. 10A-10C are AFM images of coated substrates;

FIGS. 11A-11C are SEM images of bare and coated stents showing platelet adhesion for different surfaces;

FIGS. 12A and 12B are spectral graphs obtained by DRIFT spectroscopy showing spectra obtained by DRIFT spectroscopy for a 12-amino-dodecylphosphonic acid (ADPA) monolayer;

FIG. 13 is a spectral graph obtained by DRIFT spectroscopy for pure ticagrelor overlaid with a spectral graph for ticagrelor immobilized to an ADPA monolayer on a SS316L stainless steel stent;

FIGS. 14A-14C are SEM images of surfaces of stainless steel (SS316L) stents, some of which are coated by monolayers and immobilized ticagrelor, exposed to platelet rich plasma (PRP) for one hour;

FIG. 15 are graphs showing flow cytometric platelet populations for a control stent, a bare metal stent, a stent coated by an ADPA self-assembled monolayer, and a stent with ticagrelor immobilized to the monolayer;

FIG. 16 is a bar graph showing adenosine diphosphate (ADP) Enzyme-linked immunosorbent assay (ELISA) results for the coated stents in nmol/L;

FIG. 17 is a spectral graph obtained by DRIFT spectroscopy showing immobilized ticagrelor on CoCr substrate compared to solid ticagrelor; and

FIG. 18 is a bar graph showing platelet coverage of stents surfaces for a bare metal stent and a ticagrelor coated stent.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values.

As used herein, the terms “comprising,” “comprise” or “comprised,” and variations thereof, are meant to be open ended. The terms “a” and “an” are intended to refer to one or more.

As used herein, the “treatment” or “treating” of a condition, wound, or defect means administration to a patient by any suitable dosage regimen, procedure, and/or administration route of a composition, device, or structure, with the object of achieving a desirable clinical/medical end-point, including repair and/or replacement of a tricuspid or mitral valve.

As used herein, the term “patient” or “subject” refers to members of the animal kingdom including but not limited to human beings.

A material is “biocompatible” in that the material and, where applicable, degradation products thereof, are substantially non-toxic to cells or organisms within acceptable tolerances, including substantially non-carcinogenic and substantially non-immunogenic, and are cleared or otherwise degraded in a biological system, such as an organism (patient), without substantial toxic effect. Non-limiting examples of degradation mechanisms within a biological system include chemical reactions, hydrolysis reactions, and enzymatic cleavage.

By “biodegradable” or “bioerodable,” it is meant that a material that once implanted and placed in contact with bodily fluids and tissues will degrade either partially or completely through chemical reactions with the bodily fluids and/or tissues, typically and often preferably over a time period of hours, days, weeks or months. Non-limiting examples of such chemical reactions include acid/base reactions, hydrolysis reactions, and enzymatic cleavage. The biodegradation rate of the material may be manipulated, optimized or otherwise adjusted so that the matrix degrades over a useful time period.

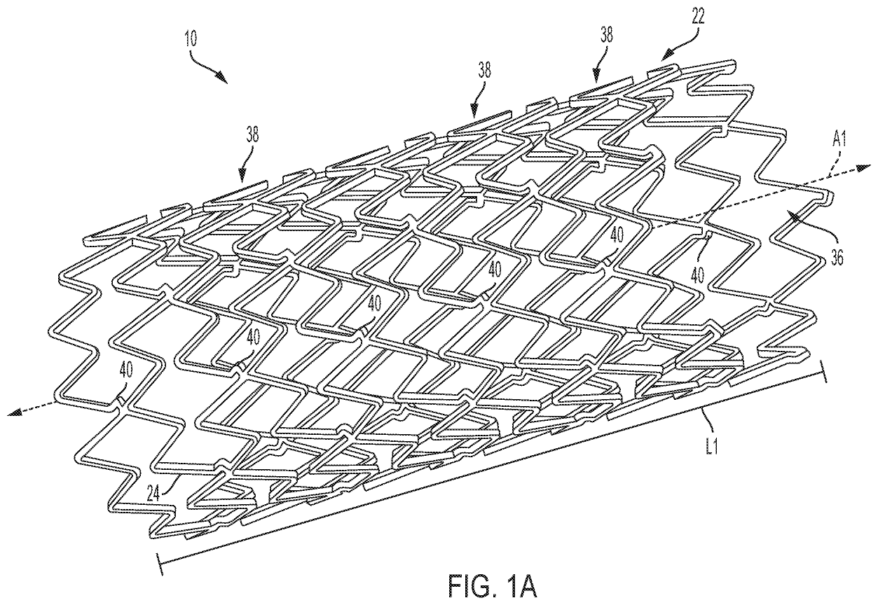

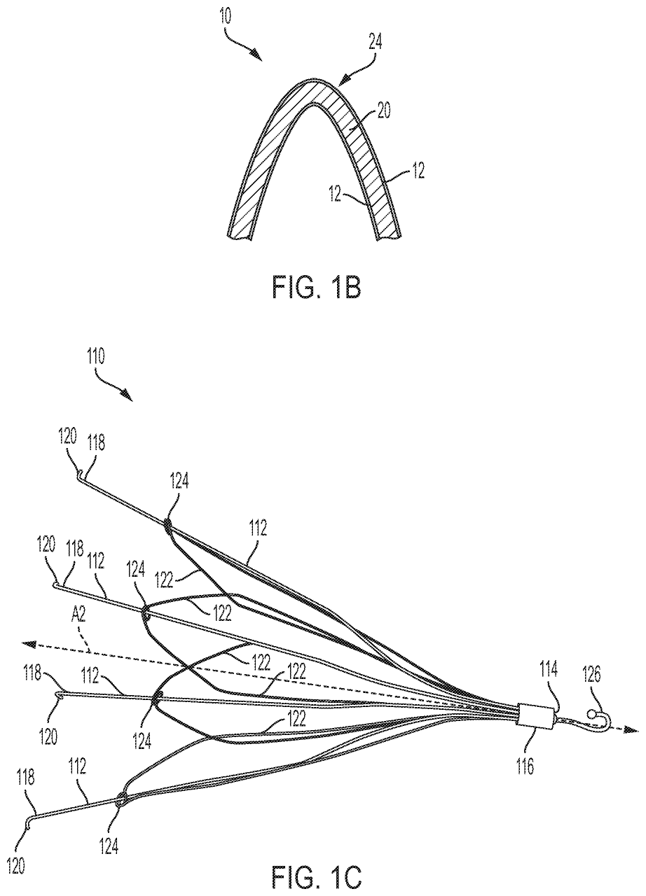

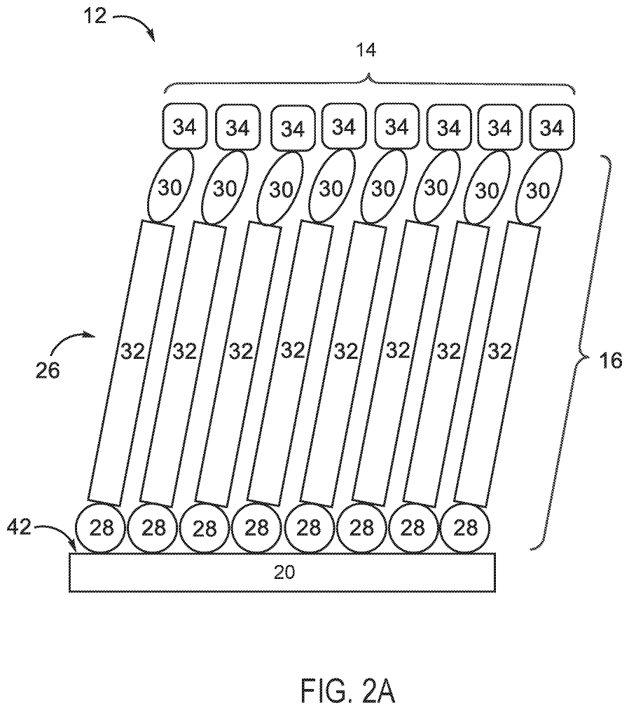

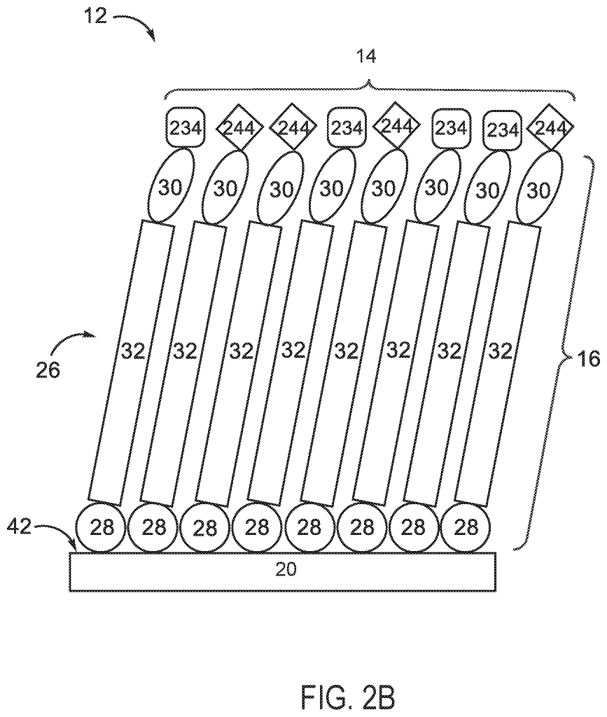

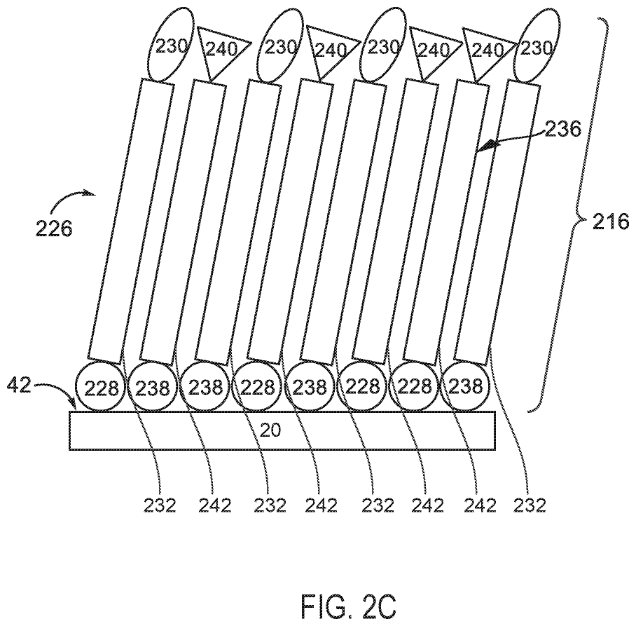

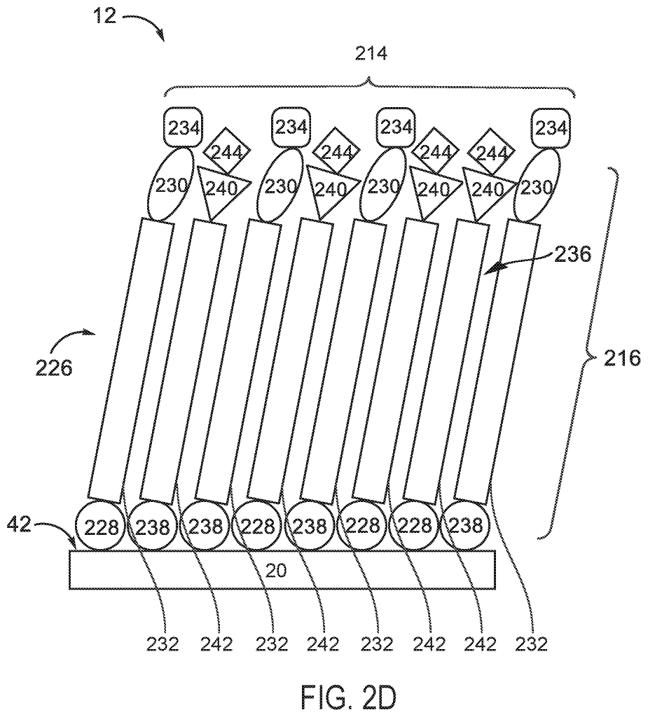

With reference to FIGS. 1A-2B, the present disclosure is generally directed to an implantable device 10 comprising a coating 12 comprising a therapeutic agent 14. As used herein, an “implantable device” can refer to various devices and structures configured to be implanted in a body of a patient. In some examples, the implantable device 10 is an endovascular device, such as a tubular stent (e.g., a venous or arterial stent), blood filter, defect or shunt closure device, intra-cardiac repair device, or fixation device that can be implanted into the vascular system of a patient through a vascular access site using a delivery catheter. A defect or shunt closure device may comprise, for example, devices configured for closing septal defects (e.g., a patent foramen ovale, atrial septal defect, or a ventricular septal defect). In other examples, the implantable device 10 can be a ventricular assist device. In other examples, the implantable device 10 can be a joint replacement device, orthopedic implant, or another blood-contacting implantable device, as are known in the art.

In some examples, the coating 12 is configured to immobilize the therapeutic agent 14 to the implantable device 10. The immobilized therapeutic agents 14 can be used to provide therapies to targeted locations surrounding the implantable device 10. Use of immobilized therapeutic agents 14, rather than materials that can be eluted from the implantable device 10, avoids potential complications and negative physiological effects of systemic delivery of antiplatelet and/or anticoagulant agents.

The implantable devices 10 and methods disclosed herein provide substantial benefits over presently available stents and treatment methods, especially relating to avoiding formation and/or treatment of stent thromboses. As discussed previously, antiplatelet drugs and antiplatelet therapies are currently used to prevent stent thrombosis. Such antiplatelet drugs can be delivered orally in sufficient concentration to provide systemic antiplatelet activity. Systemic antiplatelet therapy is non-targeted and affects an entire host platelet population. Consequently, systematic antiplatelet activity may increase bleeding. In contrast, the implantable devices 10 disclosed herein provide covalent linkages between the implantable device 10 and therapeutic agent 14, which effectively trap the antiplatelet agent to the implantable device 10.

### Self-Assembled Monolayer Examples

With reference to FIGS. 2A and 2B, the coating 12 of the implantable device 10 is formed from and/or comprises a self-assembled monolayer 16 binding the therapeutic agent 14 to a surface 42 of a body 20 (e.g., bulk material) of the implantable device 10. The body 20 is generally an elongated tubular structure. Portions of the body 20 may be formed from a suitable metal, plastic, or ceramic materials depending on the intended use of the implantable device 10. The body 20 may formed from stainless steel, for example 316L stainless steel. In other examples, portions of the body 20 may be formed from metals including titanium and/or nickel titanium alloys. The body 20 may also be formed from silicone, as is used in standard medical tubing. Silicone may be oxidized to provide bonding sites for the self-assembled monolayer 16. As described in further detail herein, the coating 12 may be formed by conventional deposition processes, such as aerosol spraying and/or immersion in a solution containing material(s) of the coating 12.

Generally, a “therapeutic agent” refers to a compound that provides a certain beneficial effect for a patient when provided to the patient in a sufficient concentration or dose. For example, the therapeutic agent 14 of the present disclosure may provide an antiplatelet therapy, such as preventing platelets from adhering to surfaces of the implantable device 10 and preventing formation of blood clots in proximity to the device 10. In other examples, the therapeutic agent 14 provides anticoagulation and/or cytotoxic properties. In some examples, as described in detail in connection with FIGS. 2B-2D, different types of therapeutic agents 14, 214 such as, for example, therapeutic agents 14, 214 having both antiplatelet and cytotoxic properties, are bonded to surfaces 42 of an implantable medical device 10. Such an implantable device 10 may be used in coronary and/or endovascular stenting to provide antiplatelet effects while simultaneously preventing neointimal hyperplasia, which may be another major mode of stent failure.

The therapeutic agent 14 may comprise ticagrelor. Ticagrelor is an antiplatelet therapy recommended by the American College of Cardiologists. As described in further detail herein, an ethanol group of a ticagrelor molecule 34 can be bonded to a tail portion 30 of a molecule 26 of the self-assembled monolayer 16. The tail portion 30 may comprise a terminal amine. As used herein, “amine” or “amino” refers to a chemical group having the indicated number of carbon atoms, where indicated, and terminating in a —NH₂ group, thus having the structure —R—NH₂, where R is an unsubstituted or substituted divalent organic group that, e.g., includes linear, branched, or cyclic hydrocarbons, and optionally comprises one or more heteroatoms.

The ticagrelor molecule(s) 34 may be bonded to the molecules 26 of the self-assembled monolayer 16 by a Mitsunobu reaction. While not intending to be bound by theory, it is believed that this Mitsunobu reaction method may be preferable because the resultant amide bond between the self-assembled monolayer 16 and ticagrelor molecules 34 are not susceptible to esterases, which would be the case when using, for example, bonds formed by ethylene diamine coupling between carboxylic acids and alcohols. The therapeutic agent 14 may comprise an anticoagulant, such as enoxaparin and fondaparinux. The therapeutic agent 14 may also comprise certain cytotoxic and/or anti-stenosis drugs designed to prevent initial hyperplasia and restenosis, as are used in conventional drug eluting stents. For example, the therapeutic agent 14 may comprise one or more of sirolimus, tacrolimus, and everolimus. In other examples, the therapeutic agent 14 can comprise a blood thinning agent, such as prasugrel. In some examples, multiple types of therapeutic agents 14 can be bonded to the self-assembled monolayer 16 to provide a variety of therapeutic effects.

With continued reference to FIG. 2A, the self-assembled monolayer 16 comprises molecules 26 that provide a linkage between a surface 42 of the implantable device 10 and the therapeutic agent 14. The self-assembled monolayer 16 may be a film or surface formed from, for example, a single layer of the molecules 26, which are vertically aligned and arranged side by side to form a substantially continuous layer.

The molecules 26 may comprise a first portion, moiety, or end (referred to hereinafter as “a head portion 28”) that spontaneously bonds to a surface of a substrate, such as a surface 42 of the body 20 of implantable device 10. As used herein, a “moiety” generally refers to a part of a molecule, and may refer to a part of a molecule that remains substantially identifiable or intact when the molecule is bonded with other compound(s) or molecule(s) to form a larger molecule, such as a polymer chain. A moiety may be a nucleotide as-incorporated into a nucleic acid or an amino acid as-incorporated into a polypeptide or protein. As used herein, “non-reactive”, in the context of a chemical constituent, such as a molecule, compound, composition, group, moiety, ion, etc., can mean that the constituent does not react with other chemical constituents in its intended use to any substantial extent. The non-reactive constituent is selected to not interfere, or to interfere insignificantly, with the intended use of the constituent, moiety, or group as a recognition reagent.

The head portion 28 may comprise an organic acid, such as phosphoric acid, carboxylic acid, bromic acid, or other organic acids capable of binding to oxygen molecules on the surface 42 of the body 20 of the implantable device 10. As used herein, an “organic acid” refers generally to an organic compound having acidic properties, which is capable of forming a covalent bond with, for example, the oxygen molecules on the surface 42. Organic acids are generally weak acids that do no dissociate in water. “Carboxyl” or “carboxylic” refers to a group having an indicated number of carbon atoms, where indicated, and terminating in a —C(O)OH group, thus having the structure —R—C(O)OH, where R is an unsubstituted or substituted divalent organic group that can include linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc. “Phosphonic” refers to a compound or molecule terminating in a —H₃PO₃ group. “Bromic” refers to a compound or molecule terminating in a —HBrO₃ group.

In one example, the molecule 26 of the self-assembled monolayer 16 is a phosphonic acid, such as 12-aminododecylphosphonic acid. In that case, the head portion 28 of the molecule 26 comprises a chemical group (e.g., a phosphonic acid group) configured to bind to the oxygen molecules on the surface 42 of the implantable device body 20. In order to ensure that the molecules 26 align in a desired orientation, the head portion 28 may be non-reactive with other surfaces and/or surfaces containing primarily other available atoms or functional groups.

In some examples, the surface 42 is used “as is” meaning that no surface preparation techniques or processes are performed on the surface 42 before the molecules 26 are bonded to the surface 42. For example, the monolayer 16 may be formed on stents provided from a manufacturer and without initial surface processing. In other examples, surfaces 42 of metal alloys can be sanded and polished using various mechanical or electrochemical techniques in order to improve surface uniformity and thus optimize binding of monolayer head portions 28, which improves surface coverage. In some examples, in order to promote spontaneous bonding with the organic acid, the surface 42 may comprise an oxidized surface and/or a surface material comprising oxygen atoms that are available for covalent bonding to the head portion 28. For example, an oxygen plasma spray preparation may be applied to the surface 42.

To provide a linkage for the therapeutic agent(s) 14, the molecules 26 of the self-assembled monolayer 16 further comprise a second portion, moiety, or end (referred to hereinafter as “the tail portion 30”) comprising one or more sites capable of reacting with reactive groups of the therapeutic agent 14 to form a suitable and sufficient covalent linkage between the molecules 26 and the therapeutic agent 14. The tail portion 30 may be non-reactive with other agents, compounds, and/or molecules during conjugation to a therapeutic agent 14 to ensure that formed monolayers 16 include a sufficient concentration of the therapeutic agent 14. The composition of the tail portion 30 may be selected based on available and/or reactive groups or moieties, such as amine, carboxyl, or thiol groups, of the therapeutic agent 14 being immobilized on the implantable device 10. The tail portion 30 may be, for example, an amine group. In other examples, the tail portion 30 may comprise a carboxyl group.

The molecule 26 of the self-assembled monolayer 16 further comprises a linker or linkage portion 32 extending between the head portion 28 and the tail portion 30 of the molecule 26. In the context of the linker moieties or linkage portions described herein, the constituents of the linkage portion 32 may be non-reactive in that they do not interfere with the binding of the head portion 28 and tail portion 30 of the molecule 26.

The linkage portion 32 may comprise an alkyl chain comprising a sufficient number of carbon atoms to provide separation between the surface 42 of the body 20 and the therapeutic agent 14, so that, for example, ticagrelor molecules 34 of the therapeutic agent 14 have sufficient space to bind to the tail portions 30 of the self-assembled monolayer 16. The linkage portion 32 is generally non-bulky in order to avoid sterically hindering or otherwise interfering, to any substantial extent, with the binding of the therapeutic agent 14 to the tail portion 30 of the molecules 26, and/or with binding of the head portion 28 to the surface 42. Further, the linkage portions 32 may be configured to adopt a particular configuration, such as a trans configuration, so that molecules 26 can be aligned and closely packed on the surface 42.

The alkyl chain of the linkage portion 32 may have from about 12 to about 18 carbon atoms, such as 16 carbons atoms. When shorter carbon chains are used, some carbon atoms may adopt a gauche configuration, causing the linkage portions 32 not to pack as well on the surface 42. The linkage portions 32 of all molecules 26 of the self-assembled monolayer 16 may be the same length. Alternatively, the length of the alkyl chains may vary to provide additional separation between the molecules 26, 34 and/or binding sites for the therapeutic agent 14. The alkyl chain may be linear and/or saturated hydrocarbyl, e.g., linear alkane.

As discussed previously, the linkage portion 32 may comprise an alkyl chain comprising a selected number of carbon atoms. As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group may be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted, for example, hydrocarbyl. Alkyl groups may be monovalent, divalent, or multivalent moieties.

Non-limiting examples of straight alkyl groups include methyl, ethyl, propyl, butyl, pentyl, hexyl, heptyl, octyl, nonyl, and decyl moieties. Branched alkyl groups comprise any straight alkyl group substituted with any number of alkyl groups. Non-limiting examples of branched alkyl groups comprise isopropyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group may be substituted with any number of straight, branched, or cyclic alkyl groups.

With reference to FIG. 2B, a coating 12 comprising a self-assembled monolayer 16 comprises the head portion 28 and linkage portion 32, as in previous examples. However, the monolayer 16 in FIG. 2B comprises a tail portion 30 configured to bind to different therapeutic agents, such as molecules 234 of a first therapeutic agent and molecules 244 of a second therapeutic agent. In order for both molecules 234, 244 to bind with sites of the tail portion 30, molecules 234, 244 may have similar chemistries and binding capabilities. In other examples, as described in detail in connection with FIGS. 2C and 2D, a self-assembled monolayer may be a mixed monolayer comprising monolayer molecules with different tail portions 30 that bind to different therapeutic agents.

### Exemplary Frame Structures

Having described features of the coating 12 and self-assembled monolayers 16, structural features of exemplary implantable devices 10 that can be coated with the coating 12 will now be described. As discussed previously, the coatings 12 and self-assembled monolayers 16 can be used with a variety of different types of implantable medical devices formed from a variety of materials. Accordingly, the implantable devices 10 described in connection with FIGS. 1A-1C are merely examples of types of devices to which the coatings 12 could be applied, and are not meant to limit the scope of the present disclosure.

With specific reference to FIGS. 1A and 1B, the implantable device 10 may be a coronary/endovascular stent. The body 20 of the stent comprises a frame 22 formed from interconnected elongated tines 24, such as bent pieces of wire or filament. Tines 24 may be formed from any material that can be drawn, such as metals or plastics. For example, the elongated tines 24 may be formed from, stainless steel, cobalt chromium, titanium oxide, nickel titanium oxide, and other alloys. The tines 24 may also be formed from high performance or engineered alloys, such as a super-elastic shape-memory material, such as nickel-titanium alloys (e.g., Nitinol). In some examples, the body 20 may further comprise a cover (not shown) formed from, for example, a mesh, film, or flexible sheet, extending over portions of the body 20. The coating 12 may cover all surfaces of the body 20 and/or cover of the implantable device 10. For example, as shown in FIG. 1B, both inwardly and outwardly facing surfaces of the elongated tines 24 may be covered by the coating 12. In other examples, only selected portions or surfaces of the body 20 may be coated by the self-assembled monolayer 16 and/or therapeutic agent 14.

The implantable device 10 may be configured to transition between a contracted state for delivery to a deployment location of a patient's vascular system by, for example, a delivery catheter, and a deployed state (shown in FIG. 1A). In the deployed state, the body 20 of the stent may expand radially outwardly to contact a wall of a vessel to anchor the body 20 in the deployment location. The body 20 may also expand longitudinally during deployment, thereby extending a length Li of the body 20 by a predetermined distance. When deployed, the body 20 contacts vessel walls to maintain patency of the vessels and to permit blood flow through a central lumen 36 of the body 20.

The elongated members or tines 24 may be arranged in a variety of patterns within the scope of the present disclosure. For example, the elongated members 24 may be arranged to form open cells, closed cells, expandable rings, or coils. As shown in FIG. 1A, in some examples, the body 20 may comprise a plurality of radially expandable annular rings 38 arranged or aligned in series along the longitudinal axis A1 of the body 20. The rings 38 may be connected to each other at connection points 40, thereby forming the elongated tubular structure. The rings 38 may be replaced by one or more helical coils. Portions of the body 20 may be covered by a suitable sheet or film, such as a metal mesh or fabric cover. In other examples, no covering or sheet is provided. In that case, the self-assembled monolayer 16 and therapeutic agent 14 may be directly deposited to the tines 24 of the frame 22.

### Coated Inferior Vena Cava Blood Filler

As discussed previously, the coating 12 and self-assembled monolayers 16 of the present disclosure may be applied to a variety of different types of implantable devices, in addition to the stent shown in FIGS. 1A and 1B.

With reference to FIG. 1C, in another example, the implantable device 10 may comprises an inferior vena cava (IVC) blood filter 110. As in previous examples, surfaces of the blood filter 110 may be coated by the coating 12 comprising the self-assembled monolayer 16. The coating 12 may be applied to all surfaces of the blood filter 110. Alternatively, some portions of the filter 110 may be coated with the coating 12 and other portions of the blood filter 110 may be bare or can be coated with self-assembled monolayers comprising different types of therapeutic agents and/or different types of coatings.

A blood filter 110 is a vascular filter configured to be deployed in the inferior vena cava to prevent, for example, pulmonary emboli from passing through the vascular system of a patient. The depicted blood filter 110 comprises support members 112, such as metallic tines. The support members 112 comprise a proximal end 114, enclosed within a collar 116, and a distal end 118. As shown in FIG. 1C, the support members 112 extend axially and radially outward from the collar 116, thereby forming an umbrella shaped structure. The distal end 118 of the support members 112 may comprise or may be bent to form hooks 120. The depicted hooks 120 are configured to engage a wall of a vessel, such as a wall of the inferior vena cava, to retain the filter 110 at a desired location in the inferior vena cava. The depicted filter 110 further comprises bent members 122 extending from the collar 116 and bent to form loops or arcs around the support members 112. The bent member 122 may be thinner and more flexible than the support member 112. As shown in FIG. 1C, the bent members 122 may extend axially and radially from the collar 116 and comprise a portion 124 that wraps around the support members 112. The blood filter 110, as shown, further comprises a hook 126 extending proximally from the collar 116, for retrieval and removal of the blood filer 110.

As with the previously-described stents, the blood filter 110 may be initially provided in a contracted configuration, in which the support members 112 and bent members 112 are closely compressed about the longitudinal axis A2 of the filter 110. In the contracted configuration, the blood filter 110 may be delivered to a deployed location through, for example, a delivery catheter, as are known in the art. Once deployed from the delivery catheter, the members 112, 122 of the filter 110 may be configured to extend radially outward from the longitudinal axis A2, to the deployed configuration shown in FIG. 1C.

As discussed previously, the coating 12 may be applied to some or all portions of the filter 110. For example, molecules 26 of the self-assembled monolayer 16 may be deposited on portions of the structural members 112, bent members 122, and/or collar 116, using the processes and techniques disclosed herein.

### Mixed Self-Assembled Monolayers

As discussed previously, in some examples, the coating 12 may comprise multiple therapeutic agents 214 that provide different therapeutic effects and treatments for the patient. In order to provide binding sites for different therapeutic agents 214 (shown in FIG. 2D), the self-assembled monolayer may be a mixed self-assembled monolayer 216 comprising multiple types of molecules. For example, as shown in FIGS. 2C and 2D, the mixed self-assembled monolayer 216 may comprise a first type of molecules (referred to herein as “first molecules 226”) and a second type of molecules (referred to herein as “second molecules 236”). The first molecules 226 and the second molecules 236 may be randomly dispersed on the surface 42 of the implantable device body 20. In some examples, the mixed monolayer 216 may comprise an equal amount of the first molecules 226 and the second molecules 236. In other examples, the molecules 226, 236 may be provided in different concentrations to achieve a particular therapeutic result.

Both the first molecules 226 and second molecules 226 comprise head portions 228, 238 bonded to the surface 42 of the implantable device body 20. The head portions 228, 238 often comprise the same functional or chemical groups for competition reasons, though first and second molecules 226, 236 with different head portions 228, 238 may also be used in some examples. For example, the head portions 228, 238 may comprise different types of organic acids.

The first molecules 226 and the second molecules 236 may further comprise tail portions 230, 240. Unlike in previous examples, the tail portions 230, 240 may be configured to bind to different types of therapeutic agents 214 (shown in FIG. 2D). For example, the tail portion 230 of the first molecule 226 may be configured to bind to a first therapeutic agent molecule 234, such as ticagrelor. The tail portion 240 of the second molecule 236 may be configured to bind to a second therapeutic agent molecule 244, such as, for example, a cytotoxic or anti-stenosis drug. In other examples, the second molecule 236 may be a spacer molecule configured to separate the first molecules 226 to improve bonding between the first molecules 226 and the first therapeutic agent molecule 234. In that case, the tail portion 240 of the second molecule 236 may be non-reactive or, at least, incapable of binding to therapeutic agent molecules 230, 240.

The first and second molecules 226, 236 may further comprise linkage portions 232, 242 extending between the head portions 228, 238 and the tail portions 230, 240. The first and second molecules 226, 236 may comprise similar or identical linkage portions 232, 242. For example, the linkage portions 232, 242 may be the same length and/or comprise a same number of carbon atoms, so that the tail portions 230, 240 are easily accessible for bonding with the therapeutic agent molecules 234, 244.

### Method of Forming a Coated Implantable Device

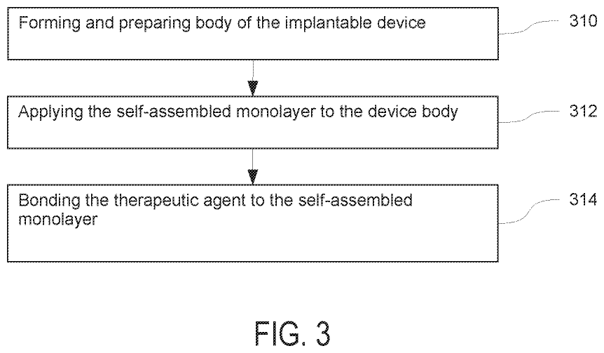

Having described embodiments of the implantable device 10 and coating 12, methods of forming such devices 10 will now be described in detail. As shown in FIG. 3, the method may comprise a step 310 of forming and/or preparing a body 20 of an implantable device 10, such as the stent (shown in FIGS. 1A and 1B) or the IVC filter (shown in FIG. 1C). As discussed previously, the body 20 comprises material(s) that react with and act as a substrate for the self-assembled monolayer 16 or mixed monolayer 216. For example, an implantable device 10 may be formed from interconnected metal tines 24 formed from stainless steel and comprising an oxidized outer surface. The tines 24 may be connected together by various processes, as are known in the art, such as welding. In other examples, a stent body 20 may be cut from a single tube of flexible metal. For example, various automated laser cutting techniques may be used to cut a stent body 20 including features, such as rings, helices, and longitudinally extending struts. Preparing the body 20 of the implantable device 10 may also comprise preparing a surface of the body 20 to bond with the self-assembled monolayers 16, 216. For example, portions of the body 20 may be oxidized to ensure that a sufficient concentration of oxygen atoms is available to bond with the self-assembled monolayer 16. Surfaces of the body 20 may also be sanded or cleaned to prepare for formation of the self-assembled monolayer 16.

At step 312, the method may further comprises applying a solution comprising molecules that form the self-assembled monolayer layer 16 or mixed monolayer 216 on surfaces of the body 20 of the implantable device 10. The solution may be applied by, for example, aerosol spraying. Alternatively, the body 20 of the implantable device 10 may be immersed in the solution containing molecules 26, 226, 236 of the self-assembled monolayers 16, 216 for a sufficient period of time to allow the self-assembled monolayers 16, 216 to form. As discussed previously, the body 20 may comprise elongated members or tines 24 formed from, for example, stainless steel. Molecules 26, 226, 236 in the solution may bind to oxygen molecules on surfaces of the body 20 of the implantable device 10 to form the self-assembled monolayers 16, 216.

Following formation of the monolayers 16, 216, at step 314, the therapeutic agent 14 may be bonded to the self-assembled monolayer 16, 216. For example, the device 10 coated by the self-assembled monolayer 16, 216 may be immersed in a solution containing molecules 34, 234, 244 of the therapeutic agent 14, 214 for a sufficient time and under suitable conditions to allow the therapeutic agent 14, 214 to bind to sites on the self-assembled monolayer 16, 216. Specifically, the therapeutic agent 14, 214 may be selected to include at least one site configured to covalently bond to the at least one site of the self-assembled monolayer 16, 216.

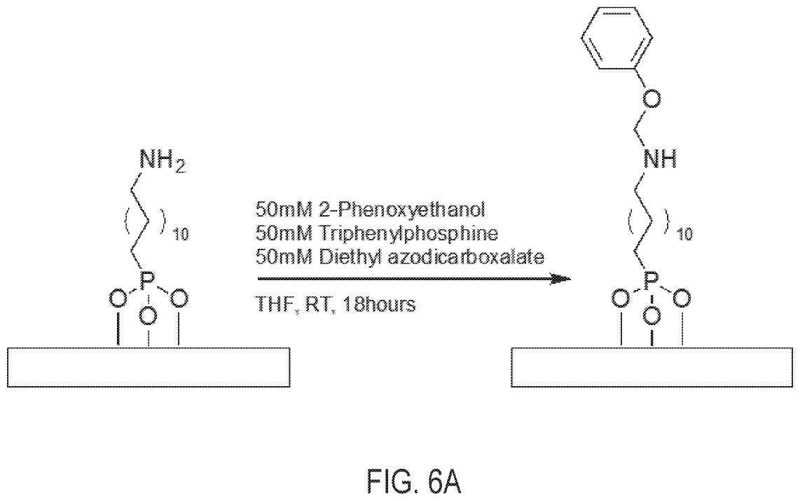

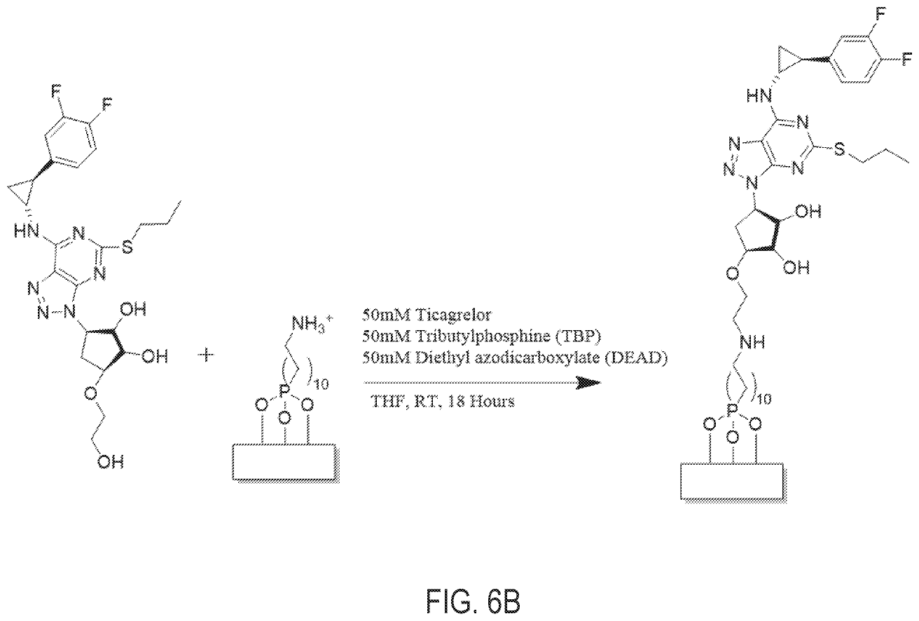

As discussed previously, in one example, the body 20 comprises 316L stainless steel, the self-assembled monolayer 16, 216 is formed from phosphonic acid (e.g., 12-aminododecylphosphonic acid), and the therapeutic agent 14, 214 comprises ticagrelor. In such an arrangement, the self-assembled monolayer 16, 216 connects to molecules 34 of the ticagrelor by an amide bond, as shown schematically in FIGS. 2A-2D and FIG. 6B. It is believed that this amide bond formation reaction provides a strong linkage between the drug to be delivered and the monolayer 16, 216. In particular, it is believed, without any intent to be bound thereby, that the monolayer 16, 216 is strongly adhered to the surface through the phosphonic acid head group. Therefore, the entire system is strongly adhered to the 316L stainless steel substrate.

### EXAMPLES

### Example 1

Monolayers composed of various phosphonic acids were synthesized on substrates to model effects of the coatings of the present disclosure on implantable devices. The monolayers all had a phosphonic acid head group and varying tail groups to allow for different organic reactions for immobilization of a therapeutic agent, such as ticagrelor, at an interface between the self-assembled monolayer and the ticagrelor.

Preparation of Planar Substrates

Initially, planar substrates of 316L stainless steel produced by Goodfellow Inc., were prepared. Specifically, substrates were cut into 1 cm×1 cm coupons. The coupons were mechanically sanded with 1200 grit sandpaper on a standard metal polisher. Progressively finer grit sandpaper was used until the substrates were polished with a 1 micron diamond suspension. The coupons were also rinsed in methanol to remove silicon carbide paper.

Formation of Self-Assembled Monolayers and Deposition of Therapeutic Agents

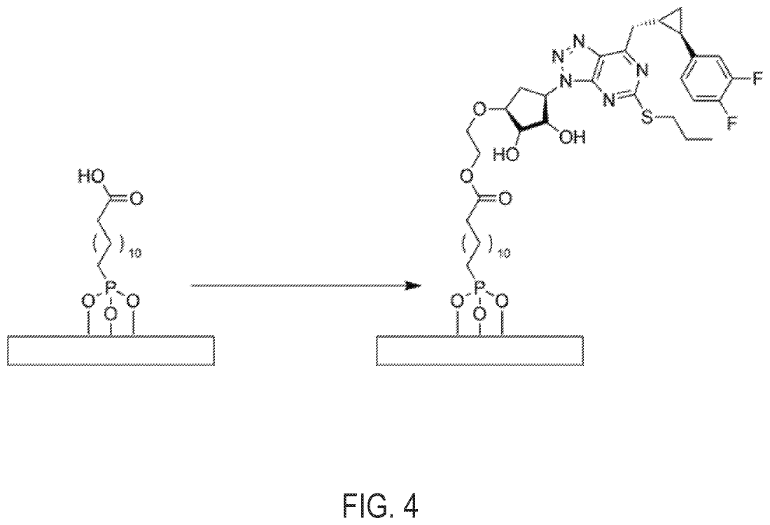

Once the substrate coupons were prepared, a monolayer composed entirely of 16-carboxyhexadecylphosphonic acid, with carboxylic acid at the interface, was synthesized and deposited on the substrate. The monolayer was deposited by aerosol spraying a 1 mM solution of 16-carboxyhexadecylphosphonic acid in tetrahydrofuran. A schematic drawing of the proposed reaction scheme using the 16-carboxyhexadecylphosphonic acid is shown in FIG. 4.

It was expected that a therapeutic agent, such as ticagrelor, could be immobilized on the monolayer formed from the 16-carboxyhexadecylphosphonic acid (“PHDA”) using ester bond formation. However, after many (>100) attempts under varying conditions, it was determined that the tail groups of the monolayer were too strongly hydrogen bonded together to react with the coupling agents effectively.

In order to address the strong hydrogen bonding between the tail groups, in a second example, spacer molecules of tetradecylphosphonic acid were placed in the monolayer at a ratio of 9:1 (16-carboxyphosphonic acid:tetradecylphosphonic acid) to form a mixed self-assembled monolayer. The self-assembled monolayers comprising the 9:1 ratio formed easily by aerosol spraying. Specifically, aerosol spraying was performed using a room temperature solution of 0.9 mM solution of 16-carboxyhexadecylphosphonic acid and 0.1 mM tetracedylphosphonic acid. The solution was sprayed onto the 316L stainless steel coupons and dried for 2 hours.

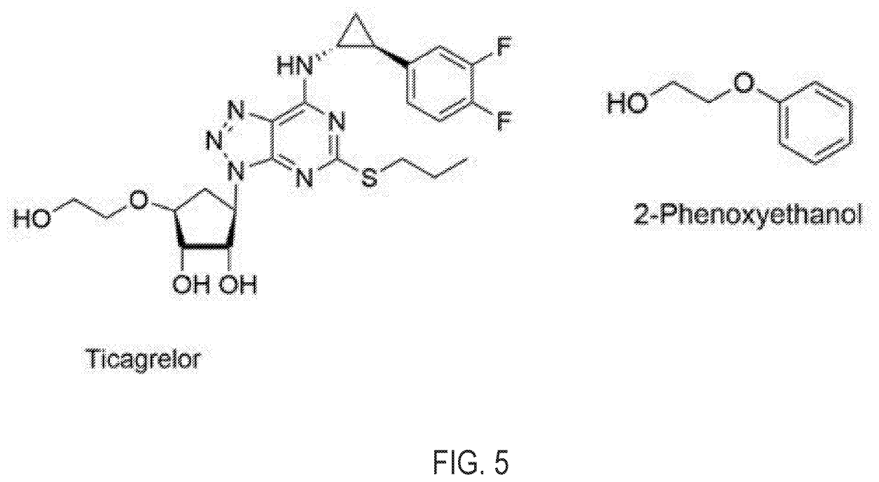

A model compound of 2-phenoxyethanol was used for ticagrelor. The 2-phenoxyethanol molecule was deemed to be an appropriate substitute because it has an identical synthetic target, specifically an ethanol tail on a ring species. A comparison of a chemical structure of tricagrelor and 2-phenoxyethanol is shown in FIG. 5. A number of different coupling reagents have been utilized, including thionyl chloride, carbodiimide cross-linking chemistry with 1-ethyl-3-(3-dimethylaminopropyl)carbodiimide (“EDC”) and N-hydroxysuccinimide (“NHS”), carbodiimide cross-linking chemistry with -dicycloexylcarbodiimde (“DCC”) and NHS, and carbodiimide cross-linking chemistry with DCC and 4-dimethylaminopyridine (“DMAP”). Example conditions can be found in the following Table. It was determined that none of the reactions worked with the carboxylic acid terminated monolayer.

TABLE
Attempted immobilization conditions for 2-Phenoxyethanol

| | Target | Crosslinking | Time | | | | |
|---|---|---|---|---|---|---|---|
| SAM | molecule | Agents | in soln | | Dry | Rinse | Solvent |
| PHDA | 1 mM | 20 mM EDC/50 | 1 | Hr | Vac | EtOH | Dry THF |
| | 2-Phen | mMNHS | | | Line | | |
| PHDA | 1 mM | 20 mM EDC/50 | 1.5 | Hr | Vac | EtOH | Dry THF |
| | 2-Phen | mMNHS | | | Line | | |
| PHDA | 2 mM | 2 mM EDC/2 | 24 | Hr | Vac | EtOH | 2- |
| | 2-Phen | mMNHS | | | Line | | Propanol |
| PHDA | 2 mM | 2 mM DCC/2 | 24 | Hr | Vac | EtOH | 2- |
| | 2-Phen | mMNHS | | | Line | | Propanol |
| PHDA | 2 mM | 2 mM DCC/2 | 24 | Hr | Vac | EtOH | 2- |
| | 2-Phen | mM DMAP | | | Line | | Propanol |
| PHDA | 5 mM | 5 mM EDC/5 | 1.5 | Hr | Vac | EtOH | 2- |
| | 2-Phen | mMNHS | | | Line | | Propanol |
| PHDA | 5 mM | 5 mM DCC/5 | 1.5 | Hr | Vac | EtOH | 2- |
| | 2-Phen | mM DMAP | | | Line | | Propanol |

In view of the difficulties in using carboxylic acid, the self-assembled monolayer was then changed from presenting a carboxylic acid terminated group at the interface to an amine. In this way, the Mitsunobu reaction, which is shown schematically in FIG. 6A, could be used to link the 2-phenoxyethanol to the surface in an amide bond. The amide bond is not subject to the esterase issues in the previous approach. Therefore, as described herein, an amine terminated monolayer was formed using 12-aminododecylphosphonic acid in tetrahydrofuran. The 12-aminododecylphosphonic acid self-assembled monolayer was formed by solution deposition, where the prepared SS316L coupons were submersed in a 1 mM solution for 30 min, followed by drying at 60° C. The amine tail group was used to react with 2-phenoxyethanol through a Mitsunobu reaction.

In order to perform the reaction, the stainless steel coupon containing the 12-aminododecylphosphonic acid self-assembled monolayer was placed in a room temperature solution of tetrahydrofuran with 50 mM 2-phenoxyethanol, 50 mM triphenylphosphine, and 50 mM diethyl azdodicarboxalate. The solution was stirred for 18 hours. Following the 18 hour period, it was determined that the 2-phenoxyethanol was successfully immobilized based on infrared spectroscopy data.

Following confirmation that the 2-phenoxyethanol could be bonded to the self-assembled monolayer, the Mitsunobu reaction was also successfully applied to immobilize ticagrelor to the surface of the coupons using the same conditions as for the 2-phenoxyethanol immobilization.

Characterization of Self-Assembled Monolayers

Three types of self-assembled monolayers were formed, as previously mentioned, in an attempt to immobilize the ticagrelor molecules. The first self-assembled monolayer was 16-carboxyhexadecylphosphonic acid, the second was a mixed self-assembled monolayer (tetradecylphosphonic acid and 16-carboxyphosphonic acid), and the third was 12-aminododecylphosphonic acid. The self-assembled monolayers were analyzed by diffuse reflectance infrared spectroscopy (“DRIFT”). A Nexus 470 Fourier Transform Infrared Spectrometer with the DRIFT attachment was used to characterize substrates after both monolayer formation and ticagrelor immobilization. Spectra were collected under nitrogen for 256 scans with a resolution of 4 cm⁻¹ on each sample and corrected with respect to a background reference spectrum of unmodified SS316L. The substrates were then sonicated in THF for 15 min to test the mechanical stability of the monolayers.

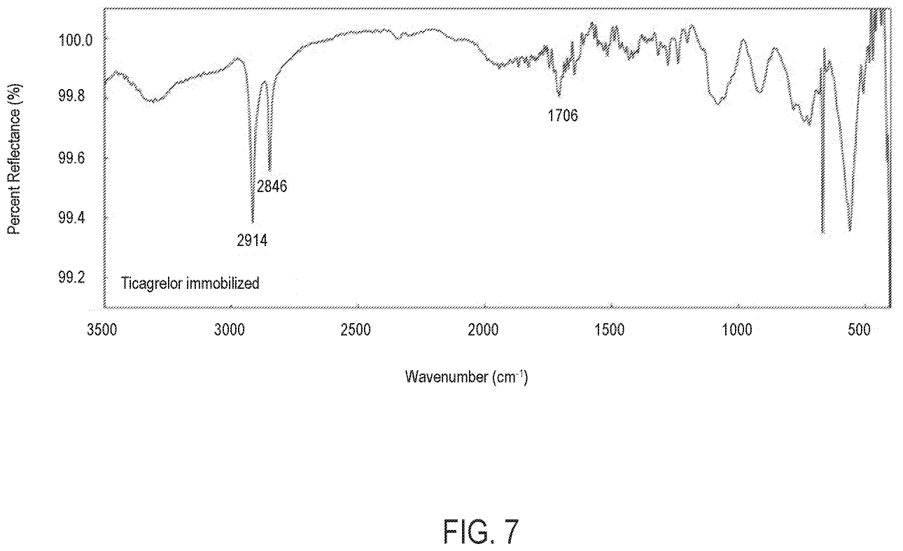

The infrared spectra indicated that all three monolayers formed. Further, using the symmetric methylene stretching (CH_{2 symm}) and asymmetric methylene stretching (CH_{2 assymm}) peaks in the infrared spectrum, the alkyl chains of the monolayers were determined to be all-trans ordered. Specifically, as shown in FIG. 7, which shows the infrared spectrum of a mixed monolayer of 16-carboxyhexadecylphosphonic acid and tetradecylphosphonic acid, the mixed self-assembled monolayer spectrum had peaks at 2914 cm⁻¹ and 2846 cm⁻¹, which are indicative of CH_{2 symm} and CH_{2 asymm} stretches, respectively, while a peak at 1706 cm⁻¹ is consistent with the carboxylic acid tail group that is not hydrogen bound. The spectra of FIG. 7 indicates that the mixed monolayers formed with all-trans alkyl chains and carboxylic acids are available at the interface for further reactions.

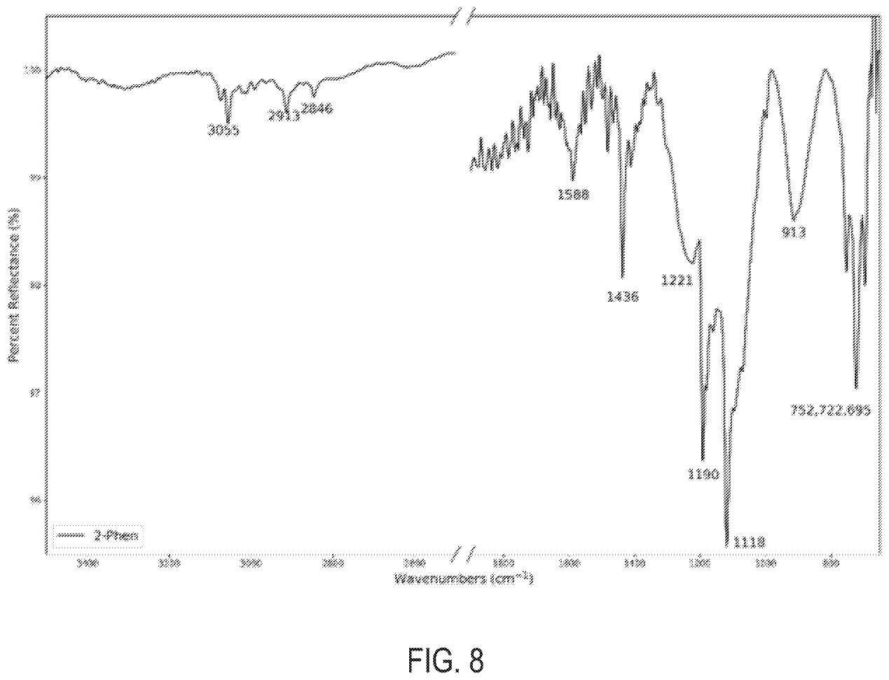

In another example, for the spectrum of 12-aminododecyl phosphonic acid, peaks indicative of the alkyl chain were determined to be at 2913 cm⁻¹ and 2846 cm⁻¹, and the free amine peak was at 1666 cm⁻¹ and 1555 cm⁻¹. The phosphonic acid head group was bonded to the surface in a tridentate manner based on the P—O group stretch at 1091 cm⁻¹. 2-phenoxyethanol was then immobilized on the surface of the 12-aminododecylphosphonic acid monolayer-modified coupons via the Mitsunobu reaction, as described previously. The spectrum for the 2-phenoxyethanol immobilized to the monolayer is shown in FIG. 8. Peaks in the spectrum in FIG. 8 are consistent with the aromatic ring (1588, 1436 cm⁻¹) in 2-phenoxyethanol. Further, the alkyl stretches were still present in the 2900 cm⁻¹ region (e.g., 2913 cm⁻¹ and 2846 cm⁻¹), which is indicative of the monolayer.

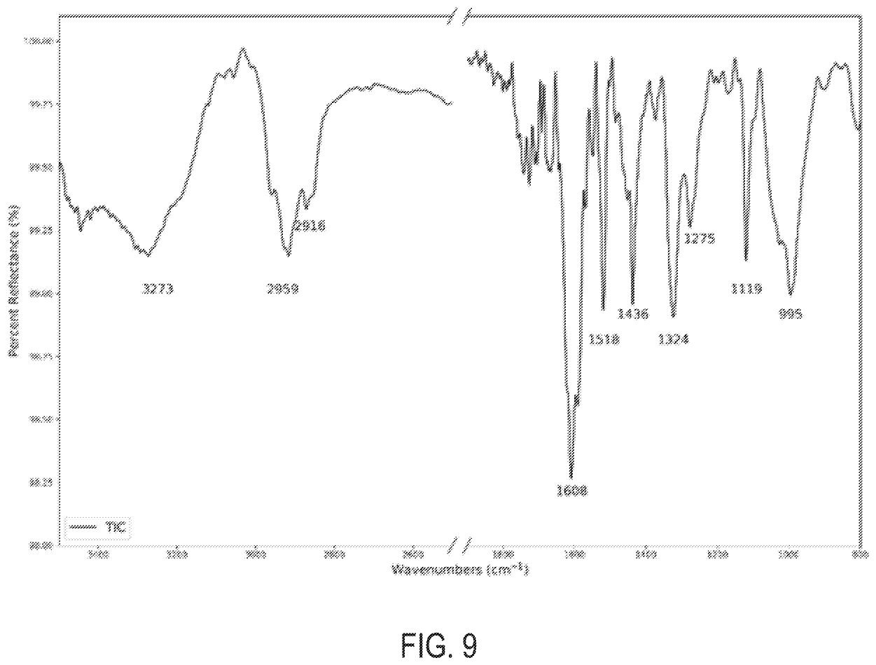

Next, ticagrelor was immobilized to coupons comprising 12-aminododecylphosphonic acid monolayer. The infrared spectra for the immobilized ticagrelor characterized by DRIFT spectroscopy is shown in FIG. 9. Peaks indicative of the ticagrelor molecule include: hydroxyl groups at 3273 cm⁻¹ and 1324 cm⁻¹; aromatic peaks at 1608 cm⁻¹, 1518 cm⁻¹, and 1436 cm⁻¹; and an aryl ether at 1324 cm⁻¹. The peaks attributed to the self-assembled monolayer linker molecule include CH₂ peaks at 2916 and 2850 cm⁻¹, and peaks in the spectrum attributed to P—O at 1119 cm⁻¹ and 995 cm⁻¹, indicating phosphonic acid bonding to the surface, as shown in FIG. 9.

AFM Characterization Protocol

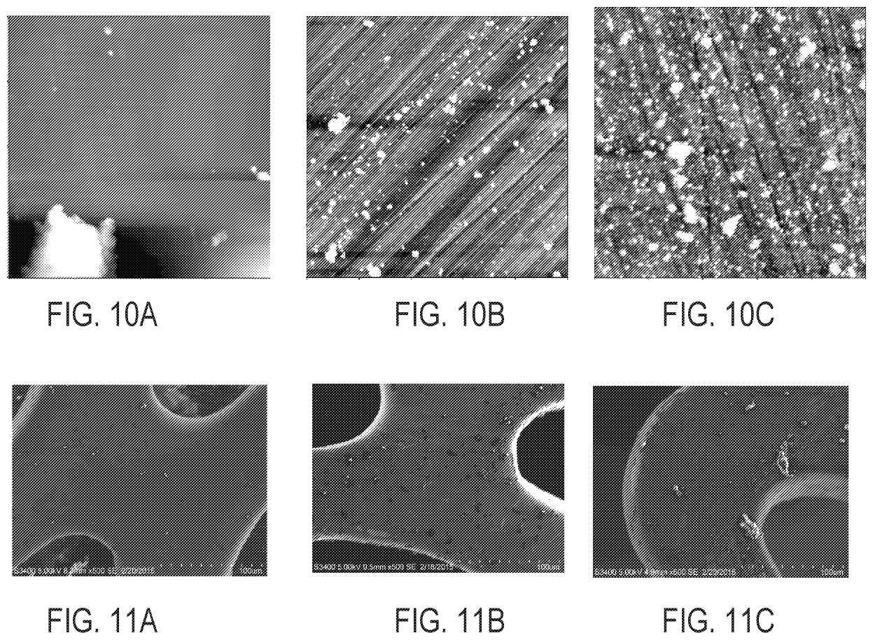

The coated coupons were also analyzed by atomic force microscopy (“AFM”). In order to perform the AFM analysis, the metal coupons were adhered to glass slides using double-sided tape. The glass slides were then mounted on a magnet and analyzed using AFM. The AFM instrument was auto-tuned, placed in phase scanning mode, centered, and then engaged the tip to the surface. The phase was ensured to be less than 90° to indicate tapping mode. Images of the coupons are shown in FIGS. 10A-10C. Particularly, FIG. 10A shows an artifact on the coupon surface representative of a bare metal stent. FIG. 10B shows a monolayer comprising 12-amino-dodecylphosphonic acid (ADPA) linker molecules. FIG. 10C shows ticagrelor molecules on the surface immobilized to the monolayer.

### Example 2

Formation of Coated Stents Formed from SS316L Stents

Monolayers of 12-amino-dodecylphosphonic acid were formed on the native oxide surface of the SS316L stents using aerosol deposition. Samples were aerosol sprayed with a 0.5 mM solution of ADPA in ethanol using thin layer chromatography sprayer, and then dried at 120° C. for 30 minutes. This process was repeated until the substrates had been sprayed 3 times, at which point the substrates were annealed at 120° C. for 24 hours. Once annealed, the substrates were rinsed and sonicated in ethanol to remove any physisorbed molecules. Monolayer formation was confirmed using contact angle goniometry, DRIFT spectroscopy, and Atomic Force Microscopy.

Self-assembled monolayer-modified stents and 0.1 g ticagrelor were placed in a flask. The flask was sealed and purged with nitrogen gas, five milliliters of dry THF was injected, followed by 60 microliters (μL) of tributylphosphosphine, after which the reaction was placed in an ice bath. Two hundred microliters of diethyl azodicarboxylate (“DEAD”) was injected via syringe pump at five microliters per minute while stirring. The reaction came to room temperature and was stirred for 48 hours. Substrates were then removed, sonicated in THF, and dried under vacuum.

Contact Angle Characterization Protocol

A Rame-Hart goniometer was used to measure static contact angles of water to determine the uniformity of the ADPA monolayers. Contact angles were measured using 2 μL drops of deionized water (Millipore 18Ω) to characterize the hydrophobicity of the surface modifications. Three measurements were performed on three substrates for a total of 9 measurements per modification. The average contact angle value and standard deviation (n=9) have been reported.

AFM Characterization Protocol

The coated stents were cut lengthwise, unfolded, and adhered to the glass slides with the double-sided tape. The glass slides were then mounted on a magnet and analyzed using AFM. The AFM instrument was auto-tuned, placed in phase scanning mode, centered, and then engaged the tip to the surface. The phase was ensured to be less than 90° to indicate tapping mode.

Platelet Adhesion Quantification and SEM Protocol

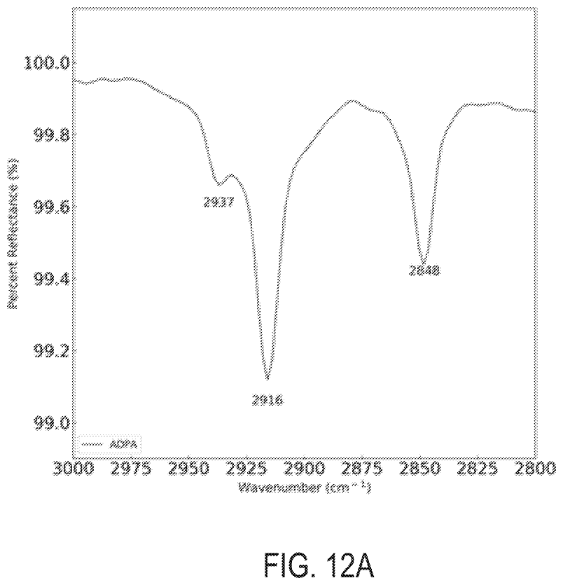

Ticagrelor molecules were immobilized on bare metal stents formed from 316L stainless steel, via deposition of a self-assembled monolayer and subsequent use of a Mitsonobu reaction. Three sets of four substrates were placed in a 24 well-plate with one set of wells left empty to act as a control. The sample sets were as follows: four empty control wells; four bare SS316L stents: four monolayer-coated substrates; and four ticagrelor-modified substrates. One milliliter of human platelet rich plasma (“PRP”, Innovative Research Inc.) was gently pipetted into each of the wells and were left for one hour at room temperature. After the time had elapsed, the unmodified and ticagrelor coated stents were removed from the PRP and placed in 5% glutaraldehyde for 7 days. The stents were then removed and submerged in 2% Osmium tetroxide (“OsO₄”) for 1 hour. The stents were then dehydrated in a series of ethanol solutions of increasing concentrations (25%, 50%, 75%, and 100%), for 20 minutes each. Dehydration was completed by final immersion in hexamethyldisilazane for 10 minutes. The stents were then desiccated for 1.5 hours prior to performing scanning electron microscopy (“SEM”). Images were taken at a magnification of 500×, working distance of 10 mm, and accelerating voltage of 5000 kV. Captured SEM images for a bare stent (FIG. 11A), an ADPA coated stent (FIG. 11B), and a stent with immobilized ticagrelor (FIG. 11C) are provided herein.

The remaining PRP was centrifuged at 3500 rpm for ten minutes to separate the remaining platelets and the supernatant for analysis. Immediately after centrifugation and the removal of the supernatant, platelet pellets were resuspended in phosphate buffered saline (PBS). The resulting platelet suspensions were incubated with both anti-human CD62P and CD42a antibodies according to manufacturer's instructions. Platelets were washed twice with PBS after incubation to remove unbound antibodies and then analyzed via flow cytometry.

Results and Discussion

Monolayers were formed from 12-aminododecylphosphonic acid to provide a functionalized amine surface for ticagrelor attachment. Self-assembled monolayers (SAMs) were formed via aerosol deposition of a 0.5 mM solution of 12-aminododecylphosphonic acid in ethanol on the oxide layer of the stents

Substrates were sonicated in ethanol to remove any physisorbed molecules and were then characterized by DRIFT spectroscopy and contact angle measurements. Stable and ordered monolayers are known to have alkyl chains in an all trans conformation, indicated by IR absorptions at ν_{CH2sym}≤2918 cm⁻¹ and ν_{CH2sym}≤2848 cm⁻¹.

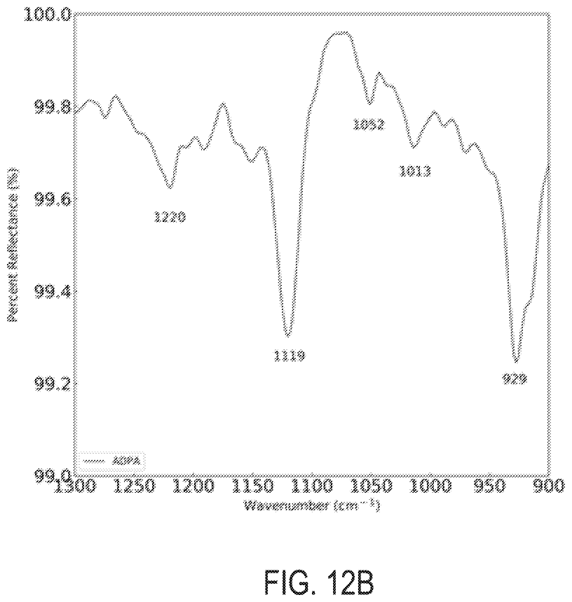

FIGS. 12A and 12B are spectral graphs showing spectra obtained by DRIFT spectroscopy for the 12-amino-dodecylphosphonic acid (ADPA) monolayer. As shown in FIG. 12A, peaks were observed at ν_{CH2asym}=2915 cm⁻¹ and ν_{CH2sym}=2848 cm⁻¹ indicating the formation of a stable and ordered SAM. An additional peak was observed at ν_{NH3+}=2937 cm⁻¹, which corresponds to the primary amine salt on the tail-group from 12-aminododecylphosphonic acid. A large peak corresponding to the C—N stretching vibration is found at ν_{C—N}=1119 cm⁻¹. The peaks at ν_{P—O}=1220 cm⁻¹, ν_{P—O}=1052 cm⁻¹ and, ν_{P—OH}=929 cm⁻¹ indicate monodentate binding of the phosphonic acid head group to the surface, as shown in FIG. 12B. The ordered alkyl chains presented the amine tail groups at the interface in a consistent manner available for further modification. The Mitsunobu reaction is a well-known method for the condensation of alcohols with various nucleophiles. Following SAM formation, tributyl phosphine was used in conjunction with diethyl azodicarboxylate (“DEAD”) to form a secondary amide cross-link between the functionalized surface and the primary hydroxyl on ticagrelor.

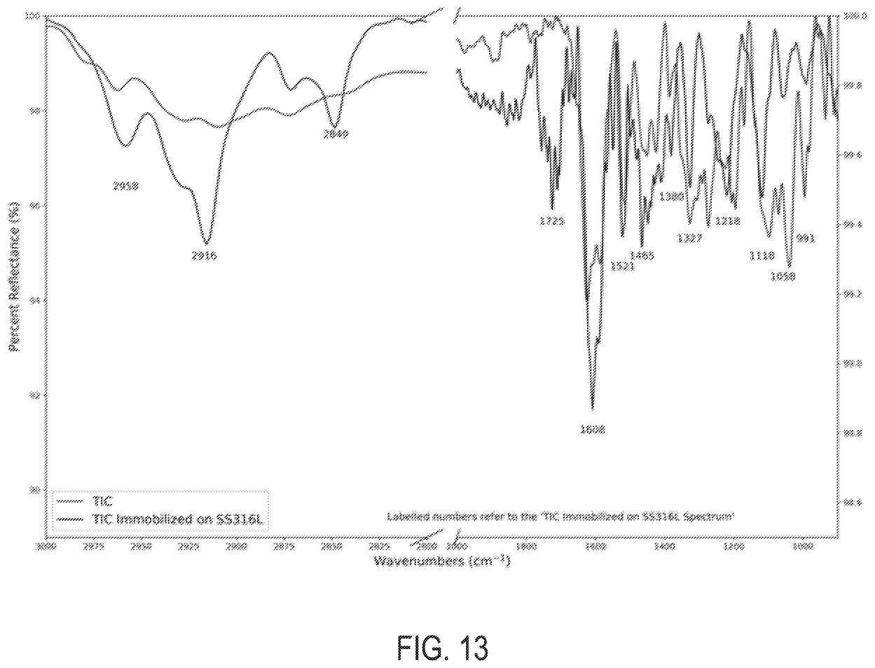

DRIFT spectra of pure ticagrelor and ticagrelor immobilized on SS316L stents were collected and overlaid to demonstrate the presence of ticagrelor on the surface. A spectral graph for the pure ticagrelor overlaid with the immobilized ticagrelor is shown in FIG. 13. As shown in FIG. 13, peaks at ν_{CH2asym}≤2916 cm⁻¹ and ν_{CH2sym}≤2849 cm⁻¹ correspond to the CH₂ symmetric and asymmetric stretching, respectively. The peak at ν_{NH3+}=2958 cm⁻¹ is due to residual unreacted primary amine salts. Indicative peaks from the immobilized ticagrelor are present at ν_{Aromatic}=1521 cm⁻¹, ν_{Aromatic}=1465 cm⁻¹, and ν_{C—N}=1327 cm⁻¹. Peaks for the asymmetric and symmetric aryl ether stretching are also present at ν_{Asym Aryl Ether}=1218 cm⁻¹ and ν_{Sym Aryl Ether}=1058 cm⁻¹. Prominent peaks can be seen at ν_{N—H Bend}=1608 cm⁻¹ and ν_{C—N Stretch}=1118 cm⁻¹, corresponding to the N—H bend and C—N stretch present on both the secondary amine on ticagrelor and the primary amine of the SAM

Static contact angle measurements were taken to evaluate changes in the wettability of the surface after SAM formation and ticagrelor immobilization. Nine measurements were collected for each sample set (3 per substrate in triplicate). Prior to reporting, the data was averaged, and the standard deviation was calculated. The contact angle of bare SS316L was found to be 72.8±4.8 degrees. The mild hydrophilicity of the surface can be attributed to the presence of oxo- and hydroxyl-groups on the native oxide layer of the metal. The contact angle measurements for the 12-aminododecylphosphonic acid coated monolayers were found to be 59.7±6.4 degrees, demonstrating a notable increase in hydrophilicity, which is expected of an amine functionalized surface. After the immobilization of ticagrelor, the contact angle was found to be 56.5±8.4 degrees. While this contact angle is effectively unchanged from that of the SAM, it is consistent with the polar structure of ticagrelor.

Once ticagrelor was immobilized on the stents a surface, a platelet challenge was used to test the efficacy of the system in the inhibition of thrombosis formation. Scanning electron microscopy (SEM), flow cytometry (FC), and an adenosine diphosphate enzyme linked immunosorbent assay (ADP ELISA) were utilized to elucidate the extent of platelet activation and aggregation on each sample. Substrates were placed into a 24 well-plate and 1 mL of platelet rich plasma (PRP) was gently pipetted into each well. The well-plate was covered and left to rest in ambient conditions for 1 hour. After exposure to the PRP, the substrates were removed and fixed in glutaraldehyde in preparation for SEM as described previously above. After the stent samples were removed, the remaining PRP from each well was centrifuged to separate the remaining platelets and the supernatant. The supernatant was removed from the platelet pellets via pipette and frozen for future analysis via ADP ELISA.

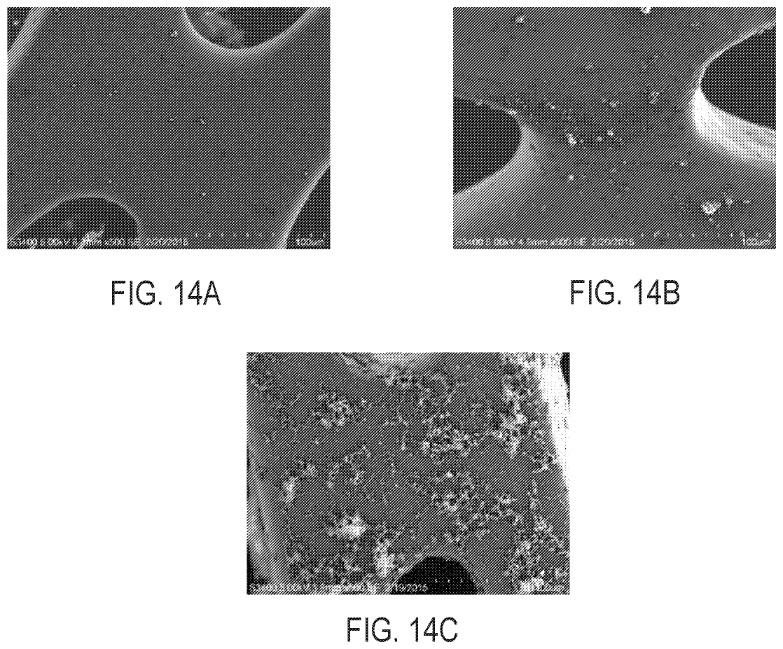

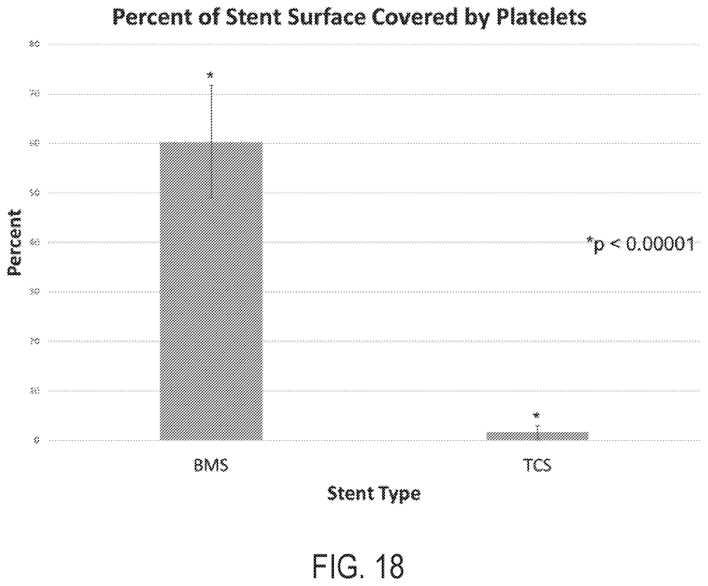

The platelet pellet was resuspended and incubated with fluorescent antibodies in preparation for measurement with the flow cytometer SEM micrographs, shown in FIGS. 14A-14C, were collected from the surface of each substrate (SS316L stents exposed to PRP for one hour) to identify changes in platelet morphology and an extent of platelet aggregation. FIG. 14A shows a bare metal stent. FIG. 14B shows an ADPA coated stent. FIG. 14C shows a ticagrelor coated stent. Platelets maintain a globular conformation prior to activation. Once activated, platelets adhere to the surface of the substrate and flatten, extending dendritic tendrils outwards to form a network with other activated platelets. The SEM micrographs of the bare SS316L surface display large aggregates of platelets, which demonstrate a fully activated morphology. Platelets adsorbed in visibly smaller quantities to 12-aminododecylphosphonic acid coated substrates. The ticagrelor immobilized surface has even less surface platelet density with maintaining a comparatively greater level of platelet aggregation after one hour of exposure to PRP than either of the other sample sets. Platelet coverage on surfaces of the stents after one hour was determined using the SEM images. As shown in FIG. 18, platelet coverage for bare metal stents was 60.33%±11.40, while platelet coverage for ticagrelor coated stents was 1.64%±1.36. Further, on both the 12-aminododecylphosphonic acid coated and ticagrelor immobilized substrates, many platelets can be seen to retain a globular morphology indicating an incomplete state of activation despite clinging to the surface.

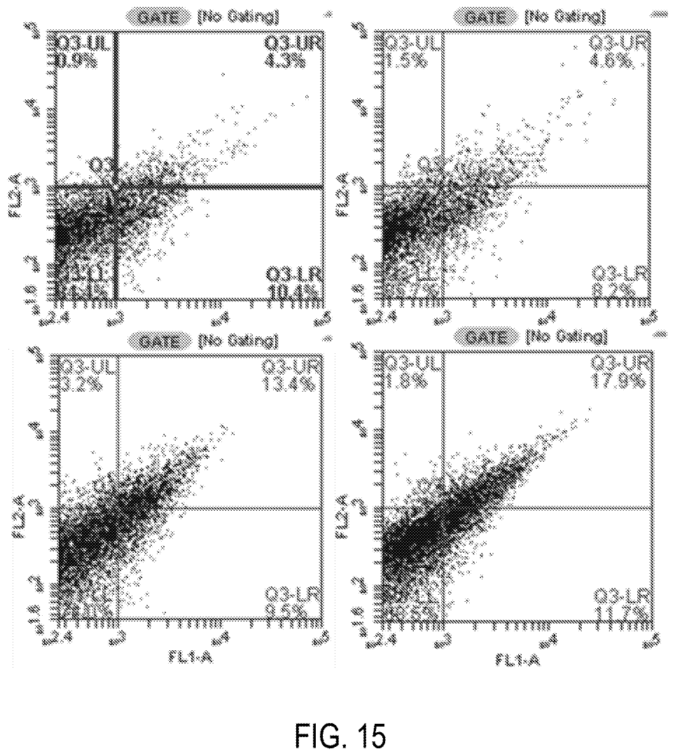

As described above, the resulting platelet suspensions were incubated with both anti-human CD62P and CD42a antibodies according to manufacturer's instructions. Platelets were washed twice with PBS after incubation to remove unbound antibodies and then analyzed via flow cytometry. The graphs in FIG. 15 show flow cytometric platelet populations for a control stent (top left), a bare metal stent (BMS) (top right), a stent coated by an ADPA self-assembled monolayer (bottom left), and a stent with ticagrelor immobilized to the monolayer (bottom right). Control and BMS samples, shown in the top row of graphs in FIG. 15, appear to have lower levels of activation (4.3% and 4.6%) than their 12-aminododecylphosphonic acid and ticagrelor coated counter-parts (13.4% and 17.9%), shown by the bottom row of graphs in FIG. 15.

It is believed that this discrepancy is a result of the large number of platelets adhered to the BMS. In that sample set, most of the activated platelets were no longer present during centrifugation, having been removed along with the substrates. With less activated platelets to bind to, it follows that those samples show less total binding by either of the fluorescence conjugated antibodies. In the case of the control samples, decreased levels of activation is expected as no foreign material is present to promote activation. It can be seen in the SEM micrographs (shown in FIGS. 14A-14C) that the ticagrelor immobilized samples (FIG. 14C) had greater levels of protection from adherence than the SAM coated samples (FIG. 14B), which is consistent with the inverse relation found in the flow cytometric data sets.

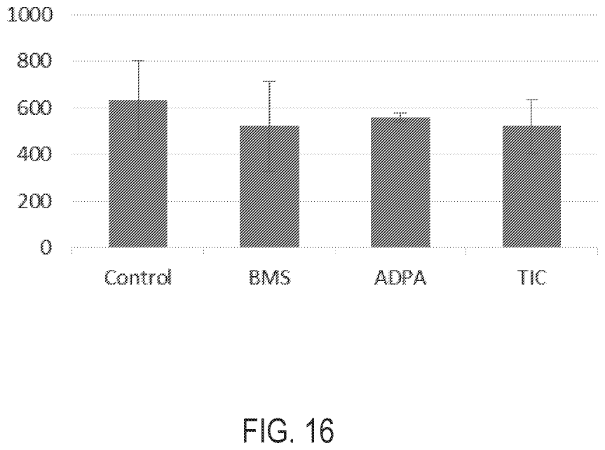

Activated platelets release Adenosine diphosphate (ADP) and other platelet activating granules as part of the coagulation. An ADP ELISA was used to investigate ADP concentration in the supernatant of each sample set following centrifugation, results of which are shown in FIG. 16. Results from the assay show that there is an equivalent amount of ADP released in each sample set and the results are shown to be significant after analysis with one-way analysis of variance (ANOVA). This indicates that, after centrifugation, all sample sets have demonstrated complete activation. These results are consistent with those of the other analyses, indicating that the surface of the substrates is protected from platelet aggregation, but does not inhibit the activation of platelets in solution. Platelets are notoriously susceptible to activation having been shown to activate upon contact with foreign materials including both polymer surfaces and fixation solvents, or even simple shear forces experienced during pipetting or centrifugation. While it is difficult to identify exactly what caused platelet activation in each of the samples, the inventors determine that the results indicate that surfaces of the ticagrelor-immobilized substrates are protected from aggregation.

### Example 3

Formation of Coated Stents Formed from Chromium Cobalt

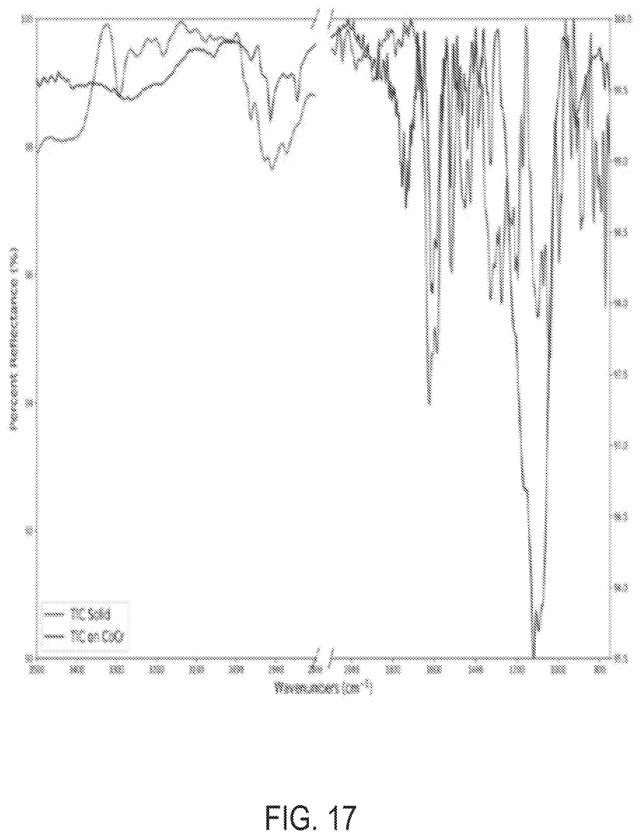

Self-assembled monolayers of 12-aminododecylphosphonic acid were formed on a CoCr substrate to show effects of the invention on different substrate materials. In order to form CoCr substrates, thin foils of CoCr were sanded using 150, 320, 400, and 600 grit sandpaper. The sanded foils were then cleaned in acetone and methanol. Bare metal stents of CoCr produced by Abbott Laboratories were also cleaned in ethanol. As in previously described examples, self-assembled monolayers were formed by single aerosol deposition of 1 mM 12-aminododecylphosphonic acid (in un-dry ethanol) onto the substrates. The coated substrates were then dried at 120° C. The substrates were then sonicated in ethanol for 15 minutes and dried for an additional 1 hour at 60° C. The amine tail group was used to link the ticagrelor molecule through a Mitsunobu reaction by stirring at room temperature in tetrahydrofuran with 50 mM ticagrelor, 50 mM triphenylphosphine, and 50 mM diethyl azdodicarboxalate. Formation of the self-assembled monolayer and immobilization of ticagrelor were characterized by DRIFT spectroscopy with peaks consistent with the ticagrelor molecule hydroxyl groups and aromatic peaks. A DRIFT spectra of immobilized ticagrelor on CoCr substrate compared to solid ticagrelor is shown in FIG. 17.

Non-limiting aspects or embodiments of the present invention will now be described in the following numbered clauses:

Clause 1: An implantable device comprising: a body configured to be implanted within a body of a patient; a self-assembled monolayer comprising molecules comprising a first portion (moiety) bonded to a surface of the body, a second portion (moiety) opposite the first portion, and a linkage portion (moiety) extending between the first portion and the second portion; and a therapeutic agent comprising at least one therapeutic molecule covalently bonded to the second portion of the molecules of the self-assembled monolayer.

Clause 2: The implantable device of clause 1, wherein the implantable device comprises a device configured to be blood contacting.

Clause 3: The implantable device of clause 2, wherein the blood-contacting device comprises at least one of a stent, filter, shunt closure device, ventricular assist device, or fixation device.

Clause 4: The implantable device of clause 1 of clause 2, wherein the blood-contacting device comprises a stent or a filter, and wherein the body of the blood-contacting device comprises a plurality of interconnected elongated members.

Clause 5: The implantable device of clause 4, wherein the plurality of interconnected members form one or more of closed or open cells, helical coils, or radially expandable rings.

Clause 6: The implantable device of any of clauses 1-5, wherein the body comprises at least one of stainless steel, cobalt chromium, titanium oxide, titanium aluminum vanadium, and nickel titanium oxide.

Clause 7: The implantable device of any of clauses 1-5, wherein the body comprises 316L stainless steel.

Clause 8: The implantable device of any of clauses 1-3, wherein the body comprises at least one of polyurethane or silicone tubing.

Clause 9: The implantable device of any of clauses 1-8, wherein the first portions of the molecules of the self-assembled monolayer comprise an organic acid, and wherein the linkage portions of the molecules comprise an alkyl chain of 12 to 18 carbon atoms, such as a linear alkane moiety.

Clause 10: The implantable device of clause 9, wherein the organic acid of the first portions of the molecules of the self-assembled monolayer comprise one or more of carboxylic acid, phosphonic acid, or bromic acid.

Clause 11: The implantable device of any of clauses 1-10, wherein the molecules of the self-assembly monolayer comprise 12-aminododecylphosphonic acid.

Clause 12: The implantable device of any of clauses 1-11, wherein the second portions of the molecules of the self-assembled monolayer comprise an amine, carboxylic acid, alcohol, thiol, methyl, or bromine

Clause 13: The implantable device of any of clauses 1-12, wherein the second portions of the molecules of the self-assembled monolayer comprise an amine.

Clause 14: The implantable device of any of clauses 1-13, wherein the therapeutic molecules comprise at least one of ticagrelor, enoaparin, fondaparinux, sirolimus, tacrolimus, everolimus, or prasugrel.

Clause 15: The implantable device of any of clauses 1-14, wherein the therapeutic agent comprises ticagrelor.

Clause 16: The implantable device of clause 15, wherein molecules of ticagrelor are covalently bonded to the second portion of the molecules of the self-assembled monolayer at an amine terminus of the molecules of the self-assembled monolayer.

Clause 17: The implantable device of any of clauses 1-16, wherein the self-assembled monolayer further comprises spacer molecules comprising tail portions that are non-reactive with the therapeutic agent.

Clause 18: The implantable device of clause 17, wherein a ratio of the molecules which are reactive with the therapeutic agent and the spacer molecules, which are non-reactive with the therapeutic agent, is about 9:1.

Clause 19: The implantable device of any of clauses 1-16, wherein the self-assembled monolayer comprises first molecules comprising tail portions configured to bind to a first type of therapeutic agent, and second molecules comprising tail portions configured to bind to a second type of therapeutic agent.

Clause 20: The implantable device of clause 19, wherein the first type of therapeutic agent comprises an anti-platelet agent, and the second type of therapeutic agent comprises a cytotoxic drug that reduces or prevents cell proliferation about the implantable device.

Clause 21: A method of deploying an implantable device, comprising: advancing the implantable device of any of clauses 1-20 through a vascular system of the patient to a preselected deployment site through a delivery catheter; and extending the implantable device from a distal end of the delivery catheter, thereby causing the implantable device to expand from a contracted state to a deployed state.

Clause 22: A method of preparing an implantable device coated by a therapeutic agent, the method comprising: preparing a body portion of an implantable device, which is configured to be blood contacting when implanted; exposing surfaces of the body of the implantable device to a solution containing molecules configured to form a self-assembled monolayer on the surfaces of the implantable device; and immersing the coated device comprising the self-assembled monolayer in a solution containing a therapeutic agent comprising at least one site configured to covalently bond to the at least one site of the self-assembled monolayer layer.

Clause 23: The method of clause 22, wherein the implantable device comprises at least one of a stent, filter, closure device, or fixation device.

Clause 24: The method of clause 22 or clause 23, wherein preparing the implantable device comprises oxidizing one or more surfaces of the implantable device to prepare the surfaces to bond to molecules of the self-assembled monolayer.

Clause 25: The method of any of clauses 22-24, wherein the body of the implantable device comprises at least one of stainless steel, cobalt chromium, titanium oxide, titanium aluminum vanadium, and nickel titanium oxide.

Clause 26: The method of clause 25, wherein the body of the implantable device comprises a plurality of interconnected radially expandable rings positioned along a longitudinal axis of the body.

Clause 27: The method of clause 22, wherein the implantable device comprises at least one of polyurethane or silicone tubing.

Clause 28: The method of any of clauses 22-27, wherein exposing the surfaces of the implantable device to the solution containing the self-assembled monolayer molecules comprises applying the solution to the surfaces by aerosol spraying.

Clause 29: The method of any of clauses 12-28, wherein molecules configured to form the self-assembled monolayer comprise a first portion comprising an organic acid bonded to the surface of the implantable device, and a linkage portion extending from the first portion comprising an alkyl chain of 12 to 18 carbon atoms, such as a linear alkane moiety.

Clause 30: The method of clause 29, wherein the organic acid of the first portions of the molecules of the self-assembled monolayer comprise at least one of carboxylic acid, phosphonic acid, or bromic acid.

Clause 31: The method of clause 29, wherein the self-assembled monolayer comprises molecules of 12-aminododecylphosphonic acid.

Clause 32: The method of any of clauses 22-31, wherein molecules of the self-assembled monolayer comprise a second portion bonded to a molecule of the therapeutic agent, the second portion comprising at least one of an amine, carboxylic acid, alcohol, thiol, methyl, or bromine.

Clause 33: The method of any of clauses 22-32, wherein the therapeutic agent comprises at least one of ticagrelor, enoaparin, fondaparinux, sirolimus, tacrolimus, everolimus, or prasugrel.

Clause 34: The method of any of clauses 22-33, wherein the therapeutic agent comprises ticagrelor, and wherein molecules of the ticagrelor are covalently bonded to molecules of the self-assembled monolayer at an amine terminus of the molecules of the self-assembled monolayer.

Clause 35: The method of any of clauses 22-34, wherein the covalent bonding of the therapeutic agent to the at least one site of the self-assembled monolayer layer occurs by a Mitsunobo reaction.

Clause 36: A method of deploying an implantable device, comprising: advancing an implantable device formed according to the method of any of clauses 22-36 through a vascular system of the patient to a preselected deployment site through a delivery catheter; and extending the implantable device from a distal end of the delivery catheter, thereby causing the implantable device to expand from a contracted state to a deployed state.

1. An implantable device comprising:
a body configured to be implanted within a body of a patient;
a self-assembled monolayer comprising molecules comprising a first portion (moiety) bonded to a surface of the body, a second portion (moiety) opposite the first portion, and a linkage portion (moiety) extending between the first portion and the second portion; and
a therapeutic agent comprising at least one therapeutic molecule covalently bonded to the second portion of the molecules of the self-assembled monolayer.

2. The implantable device of claim 1, wherein the implantable device comprises a device configured to be blood contacting, such as a stent, a filter, a shunt closure device, a ventricular assist device, or a fixation device.

3. (canceled)

4. The implantable device of claim 2, wherein the blood-contacting device comprises a stent or a filter, and wherein the body of the blood-contacting device comprises a plurality of interconnected elongated members that are optionally one or more of closed or open cells, helical coils, or radially expandable rings.

5. (canceled)

6. The implantable device of claim 1, wherein the body comprises at least one of stainless steel, cobalt chromium, titanium oxide, titanium aluminum vanadium, and/or nickel titanium oxide, and optionally comprises 316L stainless steel, and/or the body comprises at least one of polyurethane or silicone tubing.

7. (canceled)

8. (canceled)

9. The implantable device of claim 1, wherein the first portions of the molecules of the self-assembled monolayer comprise an organic acid, and wherein the linkage portions of the molecules comprise an alkyl chain of from 12 to 18 carbon atoms, such as a linear alkane moiety, wherein the organic acid of the first portions of the molecules of the self-assembled monolayer optionally comprise one or more of carboxylic acid, phosphonic acid, or bromic acid; and/or wherein the second portions of the molecules of the self-assembled monolayer optionally comprise an amine, carboxylic acid, alcohol, thiol, methyl, or bromine.

10. (canceled)

11. The implantable device of claim 1, wherein the self-assembled monolayer comprises 12-aminododecylphosphonic acid.

12. (canceled)

13. (canceled)

14. The implantable device of claim 1, wherein the therapeutic molecules comprise at least one of ticagrelor, enoaparin, fondaparinux, sirolimus, tacrolimus, everolimus, or prasugrel.

15. (canceled)

16. The implantable device of claim 14, wherein the therapeutic molecules comprise ticagrelor that optionally is covalently bonded to the second portion of the molecules of the self-assembled monolayer at an amine terminus of the molecules of the self-assembled monolayer.

17. The implantable device of claim 1, wherein the self-assembled monolayer further comprises spacer molecules comprising tail portions that are non-reactive with the therapeutic agent.

18. The implantable device of claim 17, wherein a ratio of the molecules which are reactive with the therapeutic agent and the spacer molecules, which are non-reactive with the therapeutic agent, is about 9:1.

19. The implantable device of claim 1, wherein the self-assembled monolayer comprises at least two different therapeutic molecules covalently bonded to the second portion of the molecules of the self-assembled monolayer.

20. The implantable device of claim 19, wherein the two different therapeutic molecules comprise an anti-platelet agent, and cytotoxic drug that reduces or prevents cell proliferation about the implantable device.

21. A method of deploying an implantable device, comprising:
advancing the implantable device of claim 1 through a vascular system of the patient to a preselected deployment site through a delivery catheter; and
extending the implantable device from a distal end of the delivery catheter, thereby causing the implantable device to expand from a contracted state to a deployed state.

22. A method of preparing an implantable device coated by a therapeutic agent, the method comprising:
preparing a body portion of an implantable device, which is configured to be blood contacting when implanted;
exposing surfaces of the body of the implantable device to a solution containing molecules configured to form a self-assembled monolayer on the surfaces of the implantable device; and
immersing the coated device comprising the self-assembled monolayer in a solution containing a therapeutic agent comprising at least one site configured to covalently bond to the at least one site of the self-assembled monolayer layer.

23. (canceled)

24. (canceled)

25. (canceled)

26. (canceled)

27. (canceled)

28. The method of claim 22, wherein exposing the surfaces of the implantable device to the solution containing the self-assembled monolayer molecules comprises applying the solution to the surfaces by aerosol spraying.

29. (canceled)

30. (canceled)

31. (canceled)

32. The method of claim 22, wherein molecules of the self-assembled monolayer comprise a second portion bonded to a molecule of the therapeutic agent, the second portion comprising at least one of an amine, carboxylic acid, alcohol, thiol, methyl, or bromine.

33. The method of claim 22, wherein the therapeutic agent comprises at least one of ticagrelor, enoaparin, fondaparinux, sirolimus, tacrolimus, everolimus, or prasugrel.

34. The method of claim 22, wherein the therapeutic agent comprises ticagrelor, and wherein molecules of the ticagrelor are covalently bonded to molecules of the self-assembled monolayer at an amine terminus of the molecules of the self-assembled monolayer.

35. The method of claim 22, wherein the covalent bonding of the therapeutic agent to the at least one site of the self-assembled monolayer layer occurs by a Mitsunobo reaction.

36. A method of deploying an implantable device, comprising:
advancing an implantable device formed according to the method of claim 22 through a vascular system of the patient to a preselected deployment site through a delivery catheter; and
extending the implantable device from a distal end of the delivery catheter, thereby causing the implantable device to expand from a contracted state to a deployed state.
